(19) United States
(12) Patent Application Publication
(10) Pub. No.: US 2007/0282011 A1
(43) Pub. Date: Dec. 6, 2007

(54) Biodegradable water soluble polymers

(75) Inventors: Zaza D. Gomurashvili, La Jolla, CA (US); William G. Turnell, San Diego, CA (US); Vassil Vassilev, San Diego, CA (US); Naidu Sreenivasa Chowdari, San Diego, CA (US)

Correspondence Address: DLA PIPER US LLP, 4365 EXECUTIVE DRIVE, SUITE 1100, SAN DIEGO, CA, 92121-2133, US

(73) Assignee: MediVas, LLC, San Deigo, CA (US)

(21) Appl. No.: 11/801,481

(22) Filed: May 9, 2007

Related U.S. Application Data
(60) Provisional application No. 60/799,123, filed on May 9, 2006.

(51) Int. Cl. A61K 31/12

(52) U.S. Cl. 514/687

(57) ABSTRACT

The invention provides biodegradable, water soluble PEA, PEUR and PEU carrier polymers, which can be used to conjugate, and thereby stabilize and/or solubilize, bioactive agents via polar uncharged or charged groups and activated ester or amino groups contained in the building blocks that make up the backbone of the polymer. The bioactive agents are released at a controlled rate determined by biodegradation of the polymers. The highly versatile Active Polycondensation (APC) method, which is mainly carried out in solution at mild temperatures, readily allows synthesis of such polymers. The invention water soluble polymers can also be used as water solubilizing tethers to attach drugs, and biologics to the surface of such carrier constructs as liposomes, particles and micelles.

### RELATED APPLICATIONS

This application claims priority under 35 U.S.C. §119(e) of U.S. Provisional application Ser. No. 60/799,123 filed May 9, 2006 which is hereby incorporated by reference in its entirety.

### BACKGROUND OF THE INVENTION

During the past decade, biodegradable, bioresorbable polymers for biomedical uses have garnered growing interest. Recently described, aliphatic PEAs based on α-amino acids, aliphatic diols, and fatty dicarboxylic acids have been found to be good candidates for biomedical uses because of their biocompatibility, low toxicity, and biodegradability (K. DeFife et al. Transcatheter Cardiovascular Therapeutics—TCT 2004 Conference. Poster presentation. Washington, D.C. 2004; G. Tsitlanadze, et al. J. Biomater. Sci. Polymer Edn. (2004). 15:1-24).

The highly versatile Active Polycondensation (APC) method, which is mainly carried out in solution at mild temperatures, allows synthesis of such regular, linear, polyfunctional PEAs, poly(ester-urethanes) (PEURs) and poly(ester ureas) (PEUs) with high molecular weights. Due to the synthetic versatility of APC, a wide range of material properties can be achieved in these polymers by varying the three components—α-amino-acids, diols and dicarboxylic acids—used as building blocks to fabricate the macromolecular backbone; (R. Katsarava, et al. J. Polym. Sci. Part A: Polym. Chem. (1999) 37:391-407).

It is well known that the presence of pendant hydroxyl groups enhances the biodegradability of aliphatic polymers (M. Acemoglu et al. Macromolecules (1996), 28, 3030-3037 and therein cited literature). In addition, pendant functional groups are of particular importance because they can facilitate covalent attachment of multiple bioactive agents through diverse functionalities, making, in effect, a prodrug. The pendant functional groups can also be used for attachment other functional groups.

Despite these advances in the art, there is need for new and better polymers that are biodegradable as well as soluble in water and other aqueous conditions, for example, under biological conditions, such as in blood, and the like.

### A BRIEF DESCRIPTION OF THE FIGURES

FIG. 1 is a chemical reaction scheme showing synthesis of invention negatively or positively charged water soluble polymers using protective group chemistry.

FIG. 2 is a chemical reaction scheme showing synthesis of Di-TFA salt of bis(glycine)-1,3-diglyceride (Compound 1.1).

FIG. 3 is a chemical reaction scheme showing synthesis of a Di-TFA salt of bis-(glycine)-1,2-diglyceride (Compound 1.2)

FIG. 4 is a chemical reaction scheme showing synthesis of an isomeric mixture of glycerol-bis(glycine)diester ditosylates (Compound 1.3).

### SUMMARY OF THE INVENTION

The present invention provides new biodegradable poly(ester amides) (PEAs), poly(ester urethanes) (PEURs) and poly(ester ureas) (PEUs), which are soluble in water and other aqueous conditions, for example, under biological conditions, such as in blood, and the like. The invention water soluble PEA, PEUR and PEU polymers were designed based on use of non-toxic hydrophilic residues of nontoxic, naturally occurring components or their derivatives—hydrophilic, charged or uncharged α-amino acids, glycerol or carbohydrate derived diols and short aliphatic di-acids—as building blocks to confer water solubility on the polymers.

More particularly, to yield water soluble PEAs, PEURs and PEUs, the repeat units of the polymers are composed of hydrophilic α-amino acids that are either uncharged (such as glycine, L-serine, L-threonine), positively charged (such as arginine, histidine, lysine), or negatively charged (such as aspartic and glutamic acids) and the like residues of diols or polyols (such as glycerol, dianhydrosorbitol, 1,4-anhydroerythritol, and the like) and residues of short aliphatic dicarboxylic acids (such as succinic, glutaric and diglycolic acids.

The hydrophilicity of aliphatic PEA, PEUR and PEU polymers can be varied and controlled by judicious selection of the hydrophilicity of the building blocks from which the polymer is derived. Use of monomers with pending hydrophilic groups, for example polar, but uncharged, primary or secondary hydroxyls, can increase solubility of the invention polymers in water.

Accordingly, in one embodiment the invention provides a composition comprising at least one of:

a PEA polymer having a chemical formula described by general structural formula (I), 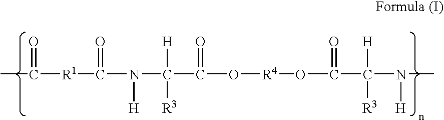
wherein n ranges from about 5 to about 150; R¹ is independently selected from (C₂-C₄) alkylene or CH₂OCH₂; R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻ and combinations thereof; and R⁴ is independently selected from the group consisting of CH₂CH(OH)CH₂ or CH₂CH(CH₂OH), bicyclic-fragments of 1,4:3,6-dianhydrohexitols of structural formula (II), residues of 1,4-anhydroerythritol and combinations thereof,

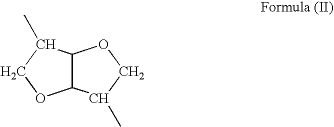

or a PEA polymer having a chemical formula described by structural formula (III): 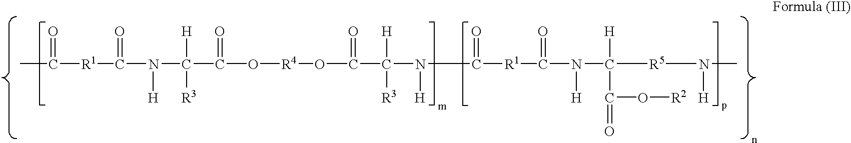
wherein n ranges from about 5 to about 150, m ranges about 0.1 to 0.9; p ranges from about 0.9 to 0.1; R¹ is independently selected from (C₂-C₄) alkylene or CH₂OCH₂; each R² is independently hydrogen, or a protecting group; the R³s in individual units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH), bicyclic-fragments of 1,4:3,6-dianhydro-hexitols of structural formula (II), residues of 1,4-anhydroerythritol and combinations thereof; and R⁵ is independently selected from the group consisting of (C₁-C₄) alkyl;

or a poly(ester urethane) (PEUR) polymer having a chemical formula described by structural formula (IV), 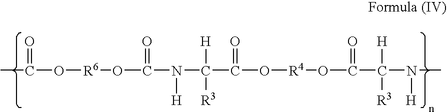
wherein n ranges from about 5 to about 150; wherein R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂, or CH₂CH(CH₂OH), bicyclic-fragments of 1,4:3,6-dianhydrohexitols of structural formula (II), residues of 1,4-anhydroerythritol, and combinations thereof; and R⁶ is independently selected from the group consisting of CH₂CH(OH)CH₂, CH₂CH(CH₂OH), residues of 1,4-anhydroerythritol, and combinations thereof;

or a PEUR polymer having a chemical structure described by general structural formula (V), 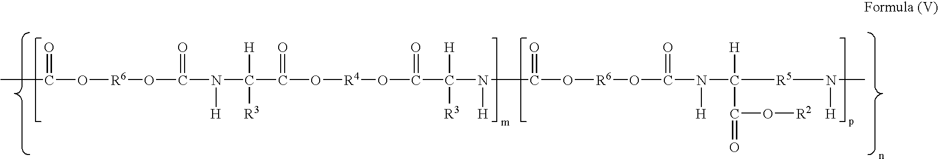
wherein n ranges from about 5 to about 150, m ranges about 0.1 to about 0.9: p ranges from about 0.9 to about 0.1; R² is independently selected from hydrogen or a protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH), bicyclic-fragments of 1,4:3,6-dianhydrohexitols of structural formula (II) residues of 1,4-anhydroerythritol, and combinations thereof; R⁶ is independently selected from the group consisting of CH₂CH(OH)CH₂, or CH₂CH(CH₂OH), residues of 1,4-anhydroerythritol, and combinations thereof; and R⁵ is independently selected from the group consisting of (C₁-C₄) alkyl;

or a poly(ester urea), (PEU) polymer having a chemical formula described by general structural formula (VI), 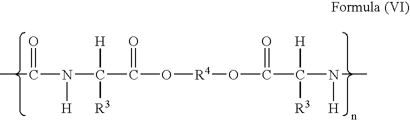
wherein n is about 10 to about 150; each R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; and R⁴ is independently selected from the group consisting of CH₂CH(OH)CH₂ or CH₂CH(CH₂OH), residues of 1,4-anhydroerythritol, and combinations thereof;

or a PEU polymer having a chemical formula described by structural formula (VII), 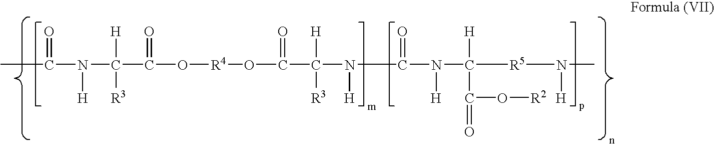
wherein m is about 0.1 to about 1.0; p is about 0.9 to about 0.1; n is about 10 to about 150; each R² is independently hydrogen, or protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazlionium, CH₂COO⁻, (CH₂)₂COO⁻, or combinations thereof; R⁴ is independently selected from the group consisting of CH₂CH(OH)CH₂ or CH₂CH(CH₂OH), residues of 1,4-anhydroerythritol, and combinations thereof; and R⁵ is independently selected from the group consisting of (C₁-C₄) alkyl, wherein the composition is biodegradable and water soluble.

### DETAILED DESCRIPTION OF THE INVENTION

The present invention is based on the discovery of new uncharged or charged aliphatic water soluble PEA polymer compositions for attachment of bioactive agents in order to solubilize a hydrophobic drug or create a favorable pharmacokinetic profile for a protein or other biologic.

Bis(α-amino acid)-α,ω-alkylene-diester is a type of diamine monomer, useful for active polycondensation (APC), inherently contains two aliphatic ester conjugations. Such ester groups can be enzymatically recognized and hydrolyzed by various esterases. Condensation of diamine monomers, for example, with active di-acid esters, results in a biodegradable PEA macromolecule with ester and amide conjugations. As di-acids, non-toxic aliphatic acids can be used. In addition, the invention PEA polymer compositions optionally can include a second monomer, such as an L-lysine based monomer, with pending C-terminus to introduce versatile properties into the polymer, such as an increase of flexibility and additional points for attachment of a bioactive agent.

The present invention provides a new type of biodegradable, water-soluble composition comprising at least one water soluble poly(ester amide) (PEA), poly(ester urethane) (PEUR) or poly(ester urea) (PEU), as described herein, as well as mixtures and blends thereof. The invention water soluble PEA, PEUR and PEU polymers were designed based on use of hydrophilic residues of nontoxic, naturally occurring components or their derivatives as building blocks to confer water solubility on the polymers.

More particularly, to yield water soluble PEAs, PEURs and PEUs, the repeat units of the polymers are composed of hydrophilic uncharged α-amino acids (such as glycine, L-serine, L-threonine, and the like), positively charged α-amino acids (arginine, histidine, lysine), negatively charged α-amino acids (aspartic and glutamic acids), diols or polyols (such as glycerol, dianhydrosorbitol, 1,4-anhydroerythritol, and the like) and short aliphatic dicarboxylic acids (such as succinic, glutaric and diglycolic acids, and the like).

The hydrophilicity of aliphatic PEA, PEUR and PEU polymers can be varied and controlled by judicious selection of the hydrophilicity of these building blocks. For example, to ensure water solubility when an uncharged α-amino acid is introduced, the other building blocks of the polymer may be selected to confer or enhance water solubility. Residues of two or three carbon diols or polyols (especially as glycerol) and residues of two or three carbon aliphatic dicarboxylic acids (e.g., succinic and glutaric acids) contribute to the water solubility of the polymer and may be used to compensate for an uncharged amino acid contained therein. The shorter the aliphatic segments in the backbone of the invention polymer compositions, the more water soluble the polymer will be. In addition, use of monomers with pending hydrophilic groups (for example, polar, but uncharged, primary or secondary hydroxyls) can increase solubility of the invention polymers in water.

In the present invention no hydrophilic moieties are conjugated to the polymers used in the invention polymer delivery compositions to make them water soluble. Instead the polymers used are of two different types. A first type has pending polar groups (uncharged or charged) existing on the monomers contained in the backbone of the polymer. A second type, which has no pending water solubilizing groups, is composed entirely of hydrophilic monomers. Both types are water soluble and stabilize an attached water soluble bioactive agent or solubilize a hydrophobic bioactive molecule conjugated thereto, making the invention polymer compositions suitable for use in biodegradable polymer delivery systems.

Accordingly, in one embodiment the invention provides a biodegradable polymer composition comprising at least one of:

a PEA polymer having a chemical formula described by general structural formula (I), 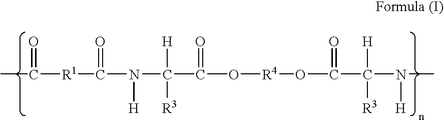
wherein n ranges from about 5 to about 150; R¹ is independently selected from (C₂-C₄) alkylene or CH₂OCH₂; R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻; (CH₂)₂COO⁻; and R⁴ is independently selected from the group consisting of CH₂CH(OH)CH₂ or CH₂CH(CH₂OH), bicyclic-fragments of 1,4:3,6-dianhydrohexitols of structural formula (II), residues of 1,4-anhydroerythritol and combinations thereof;

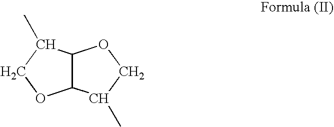

or a PEA polymer having a chemical formula described by structural formula (III): 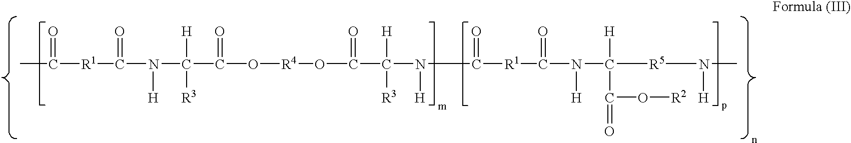
wherein n ranges from about 5 to about 150, m ranges about 0.1 to 0.9; p ranges from about 0.9 to 0.1; R¹ is independently selected from (C₂-C₄) alkylene or CH₂OCH₂; each R² is independently hydrogen, or a protecting group; the R³s in individual units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH), bicyclic-fragments of 1,4:3,6-dianhydro-hexitols of structural formula (II), residues of 1,4-anhydroerythritol and combinations thereof; and R⁵ is independently selected from the group consisting of (C₁-C₄) alkyl;

or a poly(ester urethane) (PEUR) polymer having a chemical formula described by structural formula (IV), 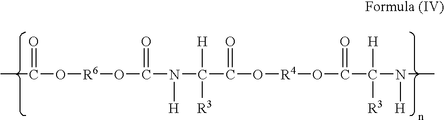
wherein n ranges from about 5 to about 150; wherein R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂, or CH₂CH(CH₂OH), bicyclic-fragments of 1,4:3,6-dianhydrohexitols of structural formula (II), residues of 1,4-anhydroerythritol, and combinations thereof; and R⁶ is independently selected from the group consisting of CH₂CH(OH)CH₂, CH₂CH(CH₂OH), residues of 1,4-anhydroerythritol, and combinations thereof;

or a PEUR polymer having a chemical structure described by general structural formula (V), 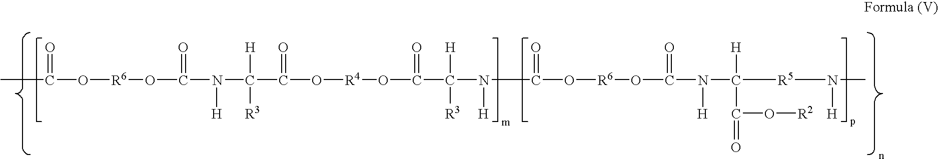
wherein n ranges from about 5 to about 150, m ranges about 0.1 to about 0.9: p ranges from about 0.9 to about 0.1; R² is independently selected from hydrogen or a protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH), bicyclic-fragments of 1,4:3,6-dianhydrohexitols of structural formula (II) residues of 1,4-anhydroerythritol, and combinations thereof; R⁶ is independently selected from the group consisting of CH₂CH(OH)CH₂, or CH₂CH(CH₂OH), residues of 1,4-anhydroerythritol, and combinations thereof; and R⁵ is independently selected from the group consisting of (C₁-C₄) alkyl; and the PEUR composition associated with counter-ions therewith, is biodegradable and water soluble.

or a poly(ester urea), (PEU) polymer having a chemical formula described by general structural formula (VI), 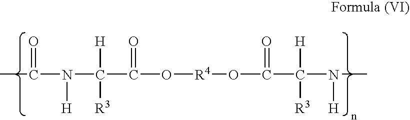
wherein n is about 10 to about 150; each R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; and R⁴ is independently selected from the group consisting of CH₂CH(OH)CH₂ or CH₂CH(CH₂OH), residues of 1,4-anhydroerythritol, and combinations thereof; and the PEU composition associated with counter-ions therewith, is biodegradable and water soluble.

or a PEU polymer having a chemical formula described by structural formula (VII), 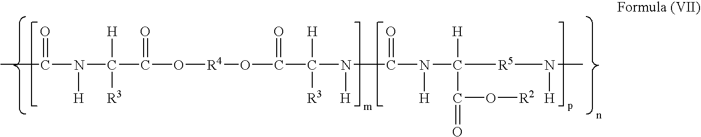
wherein m is about 0.1 to about 1.0; p is about 0.9 to about 0.1; n is about 10 to about 150; each R² is independently hydrogen, or protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, or combinations thereof; R⁴ is independently selected from the group consisting of CH₂CH(OH)CH₂ or CH₂CH(CH₂OH), residues of 1,4-anhydroerythritol, and combinations thereof; and R⁵ is independently selected from the group consisting of (C₁-C₄) alkyl, and wherein the composition is biodegradable and water soluble In certain embodiments, the polymer in the composition can have one or more counter-ions associated with charged groups therein. In other embodiments the composition can have one or more protecting groups bound to the polymer.

R⁵ in formulas (I and III-VII) is preferably (CH₂)₄ for ease of fabrication, but the number of carbons in R⁵ may be reduced to enhance water solubility of the composition.

Accordingly in another embodiment, the invention provides a biodegradable polymer composition comprising at least one bioactive agent conjugated with a PEA polymer having a chemical formula described by general structural formula (I or III), a PEUR having a chemical formula described by general structural formulas (IV or V), a PEU having a chemical formula described by general structural formulas (VI or VII), or a blend or mixture of such polymers.

Known examples of di-acids suitable for use in practice of the invention include succinic acid (when R¹ is (CH₂)₂), glutaric acid (when R¹ is (CH₂)₃), adipic acid (when R¹ is (CH₂)₄) and diglycolic acid (when R¹ is CH₂OCH₂). Succinic acid and glutaric acid are the preferred di-acids for use in preparation of invention compositions containing uncharged α-amino acids. A residue of the di-acid is incorporated into the polymer.

The bicyclic-fragments of 1,4:3,6-dianhydrohexitols also called “sugar-diols” are derived from starch, such as D-glucitol, D-mannitol, or L-iditol. For example, isosorbide (1,4:3,6-dianhydrosorbitol) and 1,4-anhydroerythritol are suitable for use in the invention water soluble polymers.

Known examples of α-amino acids suitable for incorporation into the polymers of Formulas (I and III-VII) include glycine (wherein R³ is H), L-serine (wherein R³ is CH₂OH), L-threonine (wherein R³ is CH(OH)CH₃), L-lysine (wherein R³ is (CH₂)₄NH₂), D- or L-arginine (wherein R³ is (CH₂)₃NHC(═NH)NH₂), L-histidine (wherein R³ is 4-methylene imidazole), aspartic acid (wherein R³ is CH₂COOH) and glutamic acid (wherein R³ is (CH₂)₂COOH).

Known examples of counter-ions suitable to associate with the polymer in the invention composition are cations, for example, those in bioactive agents used as therapeutics, such as Na⁺, K⁺, Ca⁺⁺, NH₄⁺, positively charged drug molecules, etc. Additionally counter-anions such are Cl⁻, F⁻, Br⁻, CH₃COO⁻, CF₃COO⁻, CCl₃COO⁻, TosO⁻, or negatively charged bioactive agents (e.g., drug molecules) can be associated with the polymer in the invention compositions.

As used herein, the terms “water solubility” and “water soluble” as applied to the invention polymer compositions means the concentration of the polymer per milliliter of deionized water at the saturation point of the polymer therein. Water solubility will be different for each different polymer, but is determined by the balance of intermolecular forces between the solvent and solute and the entropy change that accompanies the solvation. Factors such as pH, temperature and pressure will alter this balance, thus changing the solubility. The solubility is also pH, temperature, and pressure dependant.

As generally defined, water soluble polymers can include truly soluble polymers to hydrogels (G. Swift, Polymer Degr. Stab. 59: (1998) 19-24). Invention water soluble polymers can be scarcely soluble (e.g., from about 0.01 mg/mL), or can be hygroscopic and when exposed to a humid atmosphere can take up water quickly to finally form a viscous solution in which polymer/water ratio in solution can be varied infinitely.

The range of solubility of the invention polymer compositions in deionized water at atmospheric pressure is in the range from about 0.01 mg/ml to 400 mg/ml at a temperature in the range from about 18° C. to about 55° C., preferably from about 22° C. to about 40° C. Quantitative solubility of polymers can be visually estimated according to the method of Braun (D. Braun et al. in Praktikum der Makromolekularen Qrganischen Chemie, Alfred Huthig, Heidelberg, Germany, 1966). As is known to those of skill in the art, the Flory-Huggins solution theory is a theoretical model describing the solubility of polymers. The Hansen Solubility Parameters and the Hildebrand solubility parameters are empirical methods for the prediction of solubility. It is also possible to predict solubility from other physical constants such as the enthalpy of fusion.

The addition of a low molecular weight electrolyte to a solution of a polymer in deionized water can induce one of four responses. The electrolyte can cause chain contraction, chain expansion, aggregation through chelation (conformational transitions), or precipitation (phase separation). The exact nature of response will depend on various factors, such as chemical structure, concentration, molecular weight, composition of the polymer and nature of added electrolyte. Nevertheless, invention polymer compositions can be soluble in various aqueous conditions, including those found in aqueous physiological conditions, such as blood, serum, tissue, and the like.

The water solubility of the invention polymers and of conjugates of bioactive agents with the invention polymers can also be characterized using such assays as ¹H NMR, ¹³C NMR, gel permeation chromatography, and DSC as is known in the art and as illustrated in the Examples herein.

All amino acids can exist as charged species, because of the terminal amino and carboxylate groups, but only a subset of amino acids have side chains that can, under suitable conditions, be charged. An amino residue is what remains after polymerization of an amino acid monomer into a polymer, such as a protein or an invention polymer and R³ in Formulas (I and III-VII) refers to the pendant side chain of an amino acid residue.

The term “charged amino acid” as used herein to describe certain of the invention polymers, means the R³ groups therein are those of natural amino acid residues whose side chains can function as weak acids or bases—those not completely ionized when dissolved in water. The group of charged amino acids comprises arginine, aspartic acid, cysteine, glutamic acid, histidine and lysine.

The ionizable property is conferred upon these R³ groups by the presence therein of an ionizable moiety consisting of a proton that is covalently bonded to a heteroatom, which is an oxygen atom in aspartic acid, glutamic acid and tyrosine; sulfur in cysteine; and a nitrogen atom in arginine and lysine. Under suitable aqueous conditions, such as the proximity of another ionizable molecule or group, the ionizable proton dissociates from R³ as the donating hydrogen ion, rendering R³ a base which can, in turn, accept a hydrogen ion. Dissociation of the proton from the acid form, or its acceptance by the base form is strongly dependent upon the pH of the aqueous milieu. Ionization degree is also environmentally sensitive, being dependent upon the temperature and ionic strength of the aqueous mileu as well as upon the micro-environment of the ionizable group within the polymer.

Ionization constants, pK values, are tabulated below, as a guide to the relevant pH range for R³ as in natural amino acid residue X:

| X from | Ionizable group | | Charge on | |
|---|---|---|---|---|
| Amino acid | Acid | Base + H | A: acid; B: base. | pK |
| Aspartic acid | COOH | COO⁻ + H⁺ | B | 3.86 |
| Glutamic acid | COOH | COO⁻ + H⁺ | B | 4.07 |
| Histidine | NH⁺ | N + H⁺ | A | 6.10 |
| Tyrosine | OH | O⁻ + H⁺ | B | 10.0 |
| Lysine | NH₃⁺ | NH₂ + H⁺ | A | 10.5 |
| Arginine | NH₂⁺ | NH + H⁺ | A | 12.4 |

Thus, the term “charged α-amino acid” as used herein to describe certain of the invention polymers, means the R³ groups of amino acid residues therein are “chargeable”, i.e. are “ionizable” under suitable ambient aqueous conditions. Counter-ions of such charged amino acids can be examples described above and/or other bioactive agents that are ionizable under the suitable aqueous conditions.

As used herein, the term “residue of a di-acid” means that portion of a dicarboxylic-acid that excludes the two carboxyl groups of the di-acid, which portion is incorporated into the backbone of the invention polymer compositions. As used herein, the term “residue of a diol” means that portion of a diol or polyol that excludes the two hydroxyl groups thereof at the points the residue is incorporated into the backbone of the invention polymer compositions. Additional hydroxyls of a polyol can be protected or unprotected. The corresponding di-acid or diol containing the “residue” thereof is used in synthesis of the invention water soluble polymer compositions.

As used herein, the terms “α-amino acid-containing”, and “α-amino acid” mean a chemical compound containing an amino group, a carboxyl group and an R³ group as defined herein. As used herein, the terms “biological α-amino acid-containing” and “biological α-amino acid” mean the α-amino acid(s) used in synthesis are selected from glycine, L-serine, L-threonine, L-lysine, D- or L-arginine, L-histidine, aspartic and glutamic acids or a mixture thereof.

As used herein the term “bioactive agent” means an active agent that affects a biological process in a mammalian individual, such as a human, in a therapeutic or palliative manner when administered to the mammal and that is not incorporated into the polymer backbone. Bioactive agents may include, without limitation, small molecule drugs, peptides, proteins, DNA, cDNA, RNA, sugars, lipids and whole cells. One or more such bioactive agents optionally may be conjugated to the invention water soluble polymer compositions to form a prodrug for delivery of the bioactive agent in vivo at a controlled rate. For example, the bioactive agent can be delivered over a period of from about one hour to about one month. Alternatively, the bioactive agent can be tethered via the invention water soluble polymer composition to a different type of carrier construct, such as a liposome, a particle, and the like, to enhance water solubility of the conjugated bioactive agent.

In one embodiment, the PEA of structural formula (I), comprises glycerol, contains free primary and secondary pending hydroxyls, and has an alternative chemical formula described by structural formula (VIII). 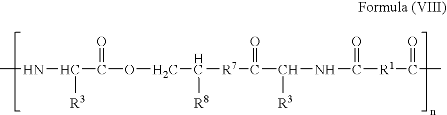
wherein n, R¹ and R³ are as above, and in each random segment when R⁷=CH₂O, then R⁸=OH, and/or when R⁷=—O—, then R⁸=CH₂OH.

The di-aryl sulfonic acid salts of diesters of α-amino acid and diol can be prepared by admixing α-amino acid, e.g., p-aryl sulfonic acid monohydrate, and diol in toluene, heating to reflux temperature, until water evolution has ceased, then cooling.

Saturated di-p-nitrophenyl esters of dicarboxylic acid and saturated di-p-toluene sulfonic acid salts of bis-α-amino acid esters can be prepared as described in U.S. Pat. No. 6,503,538 B1.

In another embodiment, the invention provides a water soluble delivery composition in which the PEA, PEUR or PEU polymer molecule has at least one bioactive agent, including drugs and biologics (denoted herein by D), attached thereto, optionally via a linker or incorporated into a crosslinker between molecules. Polymer-drug conjugations may be ester, diester, urethane, carbonate, amide, secondary or tertiary amine, ether, and the like, some of which are attached after transforming the available primary or secondary OH into —NH₂ or —SH. For example, in one embodiment, the polymer is contained in a polymer-bioactive agent conjugate having structural formula (IX): 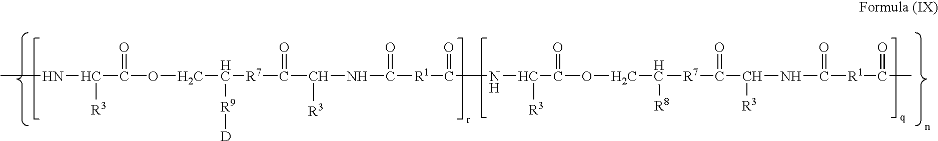
wherein n, R¹, R³, R⁷ and R⁸ are as above; r ranges from about 0.001 to about 0.9; q ranges from about 0.999 to about 0.1; except that when R⁷ is —CH₂O—, then R⁹ is —XR¹³—; and when R⁷ is —O—, then R⁹ is —CH₂XR¹³—; wherein X is a heteroatom selected from —O—, or —S—; R¹³ is selected from the group —C═O—, —COO⁻, —CO—NH—, —S—, —S(O)—, and —S(O₂)—; and D is a bioactive agent.

In yet another embodiment of the invention water soluble delivery composition, two molecules of the polymer of structural formula (IX) can be cross-linked to provide a —R⁹-D-R⁹— conjugate. In still another embodiment, as shown in structural formula (X) below, the at least one bioactive agent (e.g., a biologic) is covalently linked to two parts of a single polymer molecule of structural formula (IX) through the —R⁹-D-R⁹— conjugate, where R⁹ is as defined above;

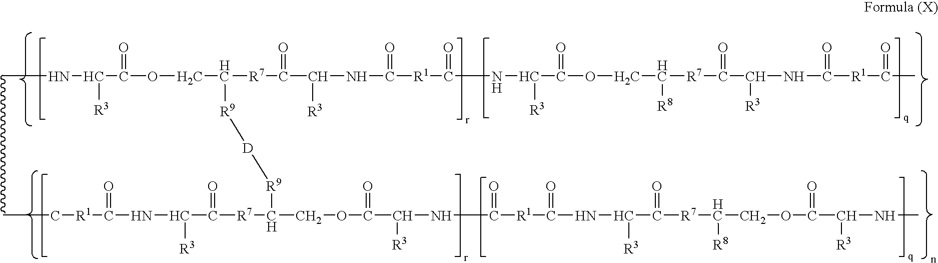

Alternatively, as shown in structural formula (XI) below, a linker, -Z-Y—, can be inserted between R⁹ and bioactive agent D, in the molecule of structural formula (VIII), wherein Z is selected from the group consisting of unsubstituted or substituted (C₁-C₈) alkylene, (C₃-C₈) cycloalkylene, 5-6 member heterocyclic system containing 1-3 heteroatoms selected from the group O, N, and S, (C₂-C₈) alkenyl, alkynyl, (C₂-C₂₀) alkyloxy (C₂-C₄)alkyl, C₆ and C₁₀ aryl, heteroaryl, alkylaryl, arylalkynyl, arylalkenyl and wherein any substituents are selected from the group consisting of H, F, Cl, Br, I, (C₁-C₆) alkyl, —CN, —NO₂, —OH, —CF₃, —O(C₁-C₄)alkyl, —S(C₁-C₆)alkyl, —S[(═O)(C₁-C₆)alkyl], —S[(O₂)(C₁-C₆)alkyl], —C[(═O)(C₁-C₆)alkyl], —O[(CO)—(C₁-C₆)alkyl], —S(O₂)[N(R¹⁴R¹⁰)], —NH[(C═O)(C₁-C₆)alkyl], —NH(C═O)N(R¹⁴R¹⁰), —N(R¹⁴R¹⁰); where R¹⁴ and R¹⁰ are independently H or (C₁-C₆) alkyl; groups as —S—, —S(O)—, —S(O₂)—, —NR¹³—, —C(═O)—, —OC(═O)—, —C(═O)O—, —OC(═O)NH—, —C(═O)NR¹¹—; and Y is selected from the group consisting of —O—, —S—, —S—S—, —S(O)—, —S(O₂)—, —NR¹¹—, —C(═O)—, —OC(═O)—, —C(═O)O—, —OC(═O)NH—, —NR¹¹C(═O)—, —C(═O)NR¹²—, —NR¹²C(═O)NR¹²—, —NR¹²C(═O)NR¹²—, and —NR¹²C(═S)NR¹²—, wherein R¹² is H or (C₁-C₈) alkyl.

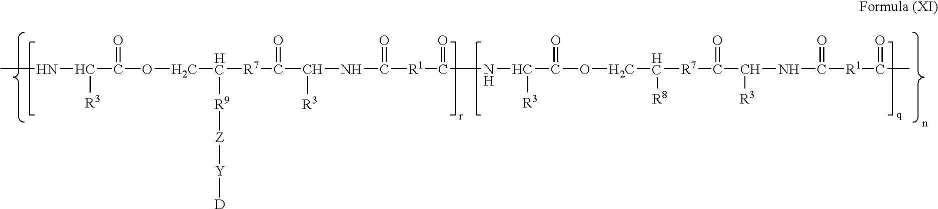

In still another embodiment, two parts of a single macromolecule are covalently linked to the bioactive agent through an —R⁹-D-Y-Z-R⁹— bridge (Formula XII): 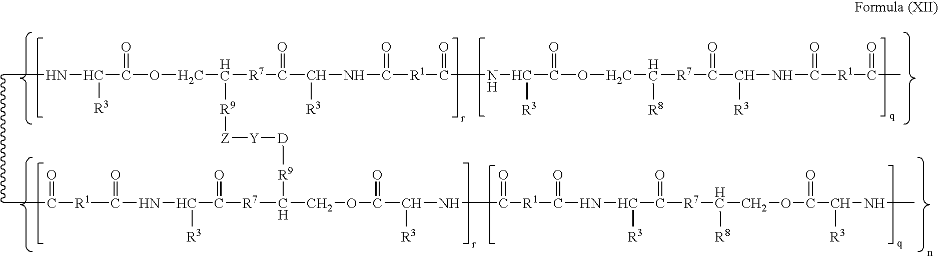
wherein, Z is selected from the group consisting of (C₁-C₈) alkylene, substituted alkylene, (C₃-C₈) cycloalkylene, substituted cycloalkylene, 5-6 membered heterocyclic system containing 1-3 heteroatoms selected from the group O, N, and S, unsubstituted and substituted heterocyclic, (C₂-C₈) alkenyl, alkynyl, (C₂-C₂₀) alkyloxy (C₂-C₄)alkyl, (C₆-C₁₀) aryl, heteroaryl, alkylaryl, arylalkynyl, arylalkenyl, wherein the substituents are selected from the group consisting of H, F, Cl, Br, I, (C₁-C₆)alkyl, —CN, —NO₂, —OH, —O(C₁-C₆)alkyl, —S(C₁-C₆) alkyl, —S[(═O)(C₁-C₆) alkyl], —S[(O₂)(C₁-C₆) alkyl], —C[(═O)(C₁-C₆)alkyl], CF₃, —O[(CO)—(C₁-C₆)alkyl], —S(O₂)[N(R¹⁴R¹⁰)], —NH[(C═O)(C₁-C₆)alkyl], —NH(C═O)N(R¹⁴R¹⁰), wherein R¹⁴ and R¹⁰ are independently H or (C₁-C₆)alkyl, and —N(R¹¹R¹²), wherein R¹¹ and R¹² are independently selected from (C₂-C₈) alkylene and (C₂-C₈) alkenylene.

Illustrative examples of “functionalizable” water-soluble biodegradable PEAs, as disclosed herein, include, but are not limited to, the following:

Glycerol based PEA Compounds 3.1-3.3 with primary, secondary or mixed pending hydroxyls:

1a. PEA, Compound 3.1, based on glycine, glycerol and adipic acid, with pending primary hydroxyls, which can be prepared from its benzylated precursor PEA Compound 3.1.1, as described herein.

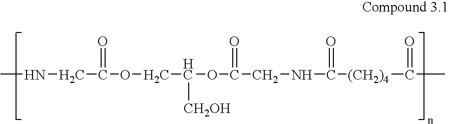

1b. PEA Compound 3.2, based on glycine, glycerol and aliphatic di-acid (adipic acid), with pending secondary hydroxyls, which can be prepared from its benzylated precursor PEA Compound 3.2.1, as described herein.

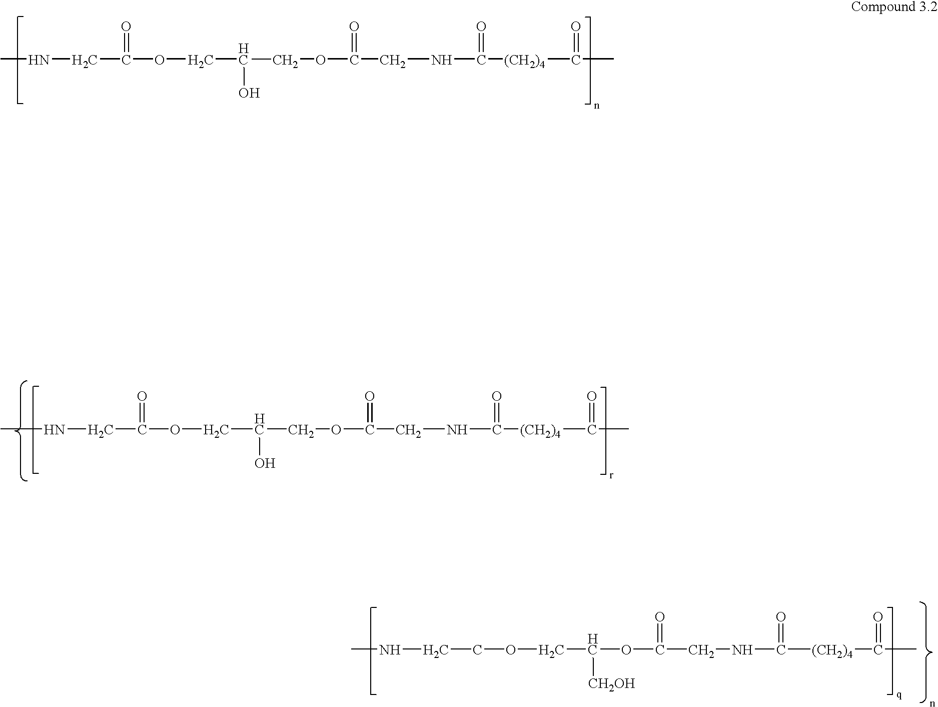

1c. PEA Compound 3.3, is a random copolymer with pending primary and secondary glycerol hydroxyls;

### Compound 3.3

Water-soluble PEA Compound 3.4, can be prepared, as described herein, from of 1,4:3,6-dianhydrosorbitol (isosorbide), glycine and succinic acid:

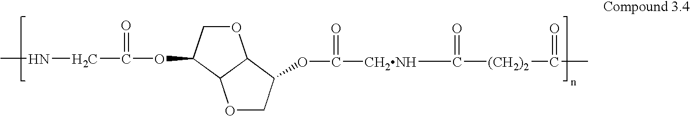

Water-soluble PEA Compound 3.5, can be prepared from 1,4-anhydroerythritol, glycine and succinic acid: 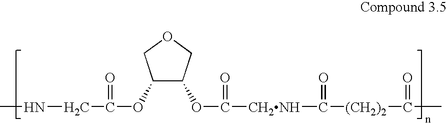
Besides increasing hydrophilicity of the invention polymers, hydroxyls also present suitable (or potential) reactive sites to modify the polymer for conjugation with bioactive agents of various types. For example, conjugation of chemotherapeutic drugs to the invention polymers is an attractive approach to reduce systemic toxicity and improve the therapeutic index of the bioactive. Polymer-drug conjugates can act as drug depots for sustained release, producing prolonged exposure of tumor cells to the chemotherapeutic drugs. In addition, the invention water soluble polymers can be used to stabilize various types of bioactive agents, as well as to solubilize otherwise insoluble bioactive agents. Thus, the invention polymers have utility as water-solubilizing carriers in targeted and site-specific drug delivery by conjugating to the polymer in at least some of the hydroxyl reactive sites in a polymer as described herein a targeting molecule as well as a therapeutic agent, such as an organic or non-organic drug molecule.

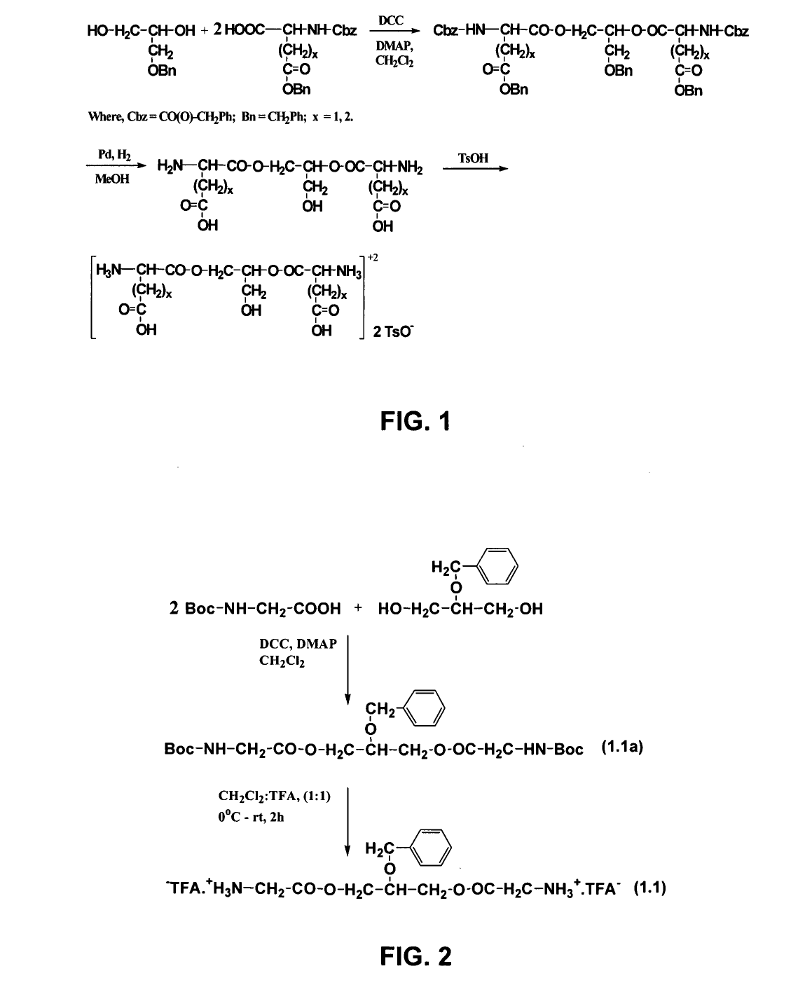

Negatively or positively charged water soluble polymers, including those containing charged α-amino acids, can be prepared using protective group chemistry. For example, bis(α-aminoacyl)-diester type monomers for synthesis of polyanion—negatively charged water soluble polymers of formulas (I and III-VII), based on aspartic or glutamic acid and glycerol can be prepared by the reaction scheme shown in FIG. 1. In this example, benzyl-protected groups were applied. Protected monomers will be de-protected either prior to APC or after polymer work-up. Suitable protective reagents and reaction conditions used in protective group chemistry can be found, e.g. in Protective Groups in Organic Chemistry, Third Edition, Greene and Wuts, Wiley & Sons, Inc. (1999), the content of which is incorporated herein by reference in its entirety.

Invention water soluble PEAs, PEURs and PEUs that lack hydroxyl groups also inherently contain functional groups at the reactive ends of the polymers suitable for the purpose of conjugation with a bioactive agent (i.e., either the amino or activated ester end-groups). Thus a bioactive agent can be readily attached at either one or both ends of the polymer macrochain to yield single or double point attachment polymers. Those of skill in the art will understand, therefore, that invention PEAs, PEURs and PEUs that lack hydroxyl groups can also readily be conjugated with a bioactive agent at the reactive ends of the polymers.

In one embodiment, the polymers used to make the invention water soluble delivery compositions as described herein have one or more bioactive agent directly linked to the polymer to form a delivery composition or prodrug for the bioactive agent. The residues of the polymer can be linked to the residues of the one or more bioactive agents. For example, one residue of the polymer can be directly linked to one residue of the bioactive agent. The polymer and the bioactive agent can each have one open valence. Alternatively, more than one bioactive agent, multiple bioactive agents, or a mixture of bioactive agents having different therapeutic or palliative activity can be directly linked to the polymer, for example through a pendant hydroxyl group or an activated ester group therein. However, since the residue of each bioactive agent can be linked to a corresponding residue of the polymer, the number of residues of the one or more bioactive agents can correspond to the number of open valences on the residue of the polymer.

As used herein, a “residue of a polymer” refers to a radical of a polymer having one or more open valences. Any synthetically feasible atom, atoms, or functional group of the polymer (e.g., on the polymer backbone or pendant group) of the present invention can be removed to provide the open valence, provided bioactivity is substantially retained when the radical is attached to a residue of a bioactive agent. Additionally, any synthetically feasible functional group (e.g., carboxyl) can be created on the polymer (e.g., on the polymer backbone or pendant group) to provide the open valence, provided bioactivity is substantially retained when the radical is attached to a residue of a bioactive agent. Based on the conjugation that is desired, those skilled in the art can select suitably functionalized starting materials that can be derived from the polymer of the present invention using procedures that are known in the art.

As used herein, a “residue of a compound of structural formula (*)” refers to a radical of a compound of polymer formulas (I) and (III-VII) as described herein having one or more open valences. Any synthetically feasible atom, atoms, or functional group of the compound (e.g., on the polymer backbone or pendant group) can be removed to provide the open valence, provided bioactivity is substantially retained when the radical is attached to a residue of a bioactive agent. Additionally, any synthetically feasible functional group (e.g., carboxyl) can be created on the compound of formulas (I) and (III-VII) (e.g., on the polymer backbone or pendant group) to provide the open valence, provided bioactivity is substantially retained when the radical is attached to a residue of a bioactive agent. Based on the conjugation that is desired, those skilled in the art can select suitably functionalized starting materials that can be derived from the compound of formulas (I) and III-VII) using procedures that are known in the art.

For example, the residue of a bioactive agent can be linked to the residue of a compound of structural formula (I) or (III-VII) through an amide (e.g., —N(R)C(═O)— or —C(═O)N(R)—), ester (e.g., —OC(═O)— or —C(═O)O—), ether (e.g., —O—), amino (e.g., —N(R)—), ketone (e.g., —C(═O)—), thioether (e.g., —S—), sulfinyl (e.g., —S(O)—), sulfonyl (e.g., —S(O)₂—), disulfide (e.g., —S—S—), or a direct (e.g., C—C bond) conjugation, wherein each R is independently H or (C₁-C₆) alkyl. Such a conjugation can be formed from suitably functionalized starting materials using synthetic procedures that are known in the art. Based on the conjugation that is desired, those skilled in the art can select suitably functional starting material that can be derived from a residue of a compound of structural formula (I) or (III-VII) and from a given residue of a bioactive agent or adjuvant using procedures that are known in the art. The residue of the bioactive agent or adjuvant can be linked to any synthetically feasible position on the residue of a compound of structural formula (I) or (III-VII). In yet another example, a bioactive agent can be linked with charged water soluble polymer of formula (I) or (III-VII) via ionic (non-covalent) interaction. Additionally, the invention also provides compounds having more than one residue of a bioactive agent or adjuvant bioactive agent directly linked to a compound of structural formula (I) or (III-VII).

The number of bioactive agents that can be linked to the polymer molecule can typically depend upon the molecular weight of the polymer. For example, for a compound of structural formula (I), wherein n is about 5 to about 150, preferably about 5 to about 70, up to about 50 bioactive agent molecules (i.e., residues thereof) can be directly linked to the polymer (i.e., residue thereof) by reacting the bioactive agent with side groups of the polymer. In unsaturated polymers, the bioactive agents can also be reacted with double (or triple) bonds in the polymer.

In other embodiments, a bioactive agent can be linked to any of the polymers of structures (I and III-VII) through an amino, hydroxyl (alcohol) or thiol conjugation. Such a conjugation can be formed from suitably functionalized starting materials using synthetic procedures that are known in the art.

For example, in one embodiment a polymer can be linked to the bioactive agent via a carboxyl group (e.g., COOH) of the polymer. Specifically, a compound of structures (I) and (III) can react with an amino functional group or a hydroxyl functional group of a bioactive agent to provide a biodegradable water soluble polymer having the bioactive agent attached via an amide conjugation or carboxylic ester conjugation, respectively. In another embodiment, the carboxyl group of the polymer can be benzylated or transformed into an acyl halide, acyl anhydride/“mixed” anhydride, or active ester. In other embodiments, the free —NH₂ ends of the polymer molecule can be acylated to assure that the bioactive agent will attach only via a carboxyl group of the polymer and not to the free ends of the polymer.

A linear polymer polypeptide conjugate is made by protecting the potential nucleophiles on the polypeptide backbone and leaving only one reactive group to be bound to the polymer or polymer linker construct. Deprotection is performed according to methods well known in the art for deprotection of peptides (Boc and Fmoc chemistry for example).

In one embodiment of the present invention, a polypeptide bioactive agent is presented as retro-inverso or partial retro-inverso peptide. Accordingly, the terms “peptide” and “polypeptide,” as used herein, include peptides, wholly peptide derivatives (such as branched peptides) and covalent hetero- (such as glyco-, lipo- and glycolipo-) derivatives of peptides.

The peptides described herein can be synthesized using any technique as is known in the art. The peptides and polypeptides can also include “peptide mimetics.” Peptide analogs are commonly used in the pharmaceutical industry as non-peptide bioactive agents with properties analogous to those of the template peptide. These types of non-peptide compound are termed “peptide mimetics” or “peptidomimetics.” Fauchere, J. (1986) Adv. Bioactive agent Res., 15:29; Veber and Freidinger (1985) TINS p. 392; and Evans et al. (1987) J. Med. Chem., 30:1229; and are usually developed with the aid of computerized molecular modeling. Generally, peptidomimetics are structurally similar to a paradigm polypeptide (i.e., a polypeptide that has a biochemical property or pharmacological activity), but have one or more peptide conjugations optionally replaced by a conjugation selected from the group consisting of —CH₂NH—, —CH₂S—, —CH₂—CH₂—, —CH═CH— (cis and trans), —COCH₂—, —CH(OH)CH₂—, and —CH₂SO—, by methods known in the art and further described in the following references: Spatola, A. F. in “Chemistry and Biochemistry of Amino Acids, Peptides, and Proteins,” B. Weinstein, eds., Marcel Dekker, New York, p. 267 (1983); Spatola, A. F., Vega Data (March 1983), Vol. 1, Issue 3, “Peptide Backbone Modifications” (general review); Morley, J. S., Trends. Pharm. Sci., (1980) pp. 463-468 (general review); Hudson, D. et al., Int. J. Pept. Prot. Res., (1979) 14:177-185 (—CH₂ NH—, —CH₂CH₂—); Spatola, A. F. et al., Life Sci., (1986) 38:1243-1249 (—CH₂—S—); Harm, M. M., J. Chem. Soc. Perkin Trans I (1982) 307-314 (—CH═CH—, cis and trans); Almquist, R. G. et al., J. Med. Chem., (1980) 23:2533 (—COCH₂—); Jennings-Whie, C. et al., Tetrahedron Lett., (1982) 23:2533 (—COCH₂—); Szelke, M. et al., European Appln., EP 45665 (1982) CA: 97:39405 (1982) (—CH(OH)CH₂—); Holladay, M. W. et al., Tetrahedron Lett., (1983) 24:4401-4404 (—C(OH)CH₂—); and Hruby, V. J., Life Sci., (1982) 31:189-199 (—CH₂—S—). Such peptide mimetics may have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others.

Additionally, substitution of one or more amino acids within a peptide or polypeptide (e.g., with a D-Lysine in place of L-Lysine) may be used to generate more stable peptides and peptides resistant to endogenous proteases. Alternatively, the synthetic peptide or polypeptide, e.g., covalently bound to the biodegradable polymer, can also be prepared from D-amino acids, referred to as inverso peptides. When a peptide is assembled in the opposite direction of the native peptide sequence, it is referred to as a retro peptide. In general, peptides prepared from D-amino acids are very stable to enzymatic hydrolysis. Many cases have been reported of preserved biological activities for retro-inverso or partial retro-inverso peptides (U.S. Pat. No. 6,261,569 B1 and references therein; B. Fromme et al, Endocrinology (2003)144:3262-3269).

Polymer—Bioactive Agent Conjugation.

Alternatively, more than one bioactive agent, multiple bioactive agents, or a mixture of bioactive agents and additional bioactive agents having different therapeutic or palliative activity can be directly linked to the polymer. However, since the residue of each bioactive agent can be linked to a corresponding residue of the polymer, the number of residues of the one or more bioactive agents can correspond to the number of open valences on the residue of the polymer.

As used herein, a “bioactive agent” refers to a therapeutic, palliative or diagnostic agent that is conjugated to the invention biodegradable water soluble polymer of structural formulas (I or III-VII) when the polymer is used as a carrier or as a tether to attach the bioactive agent to another carrier entity, such as a particle, liposome or micelle. Specifically, such additional bioactive agent can include, but is not limited to, one or more of: polynucleotides, polypeptides, oligonucleotides, gene therapy agents, nucleotide analogs, nucleoside analogs, polynucleic acid decoys, therapeutic antibodies, abciximab, anti-inflammatory agents, blood modifiers, anti-platelet agents, anti-coagulation agents, immune suppressive agents, anti-neoplastic agents, anti-cancer agents, anti-cell proliferation agents, and nitric oxide releasing agents.

The polynucleotide can include deoxyribonucleic acid (DNA), ribonucleic acid (RNA), double stranded DNA, double stranded RNA, duplex DNA/RNA, antisense polynucleotides, functional RNA or a combination thereof. In one embodiment, the polynucleotide can be RNA. In another embodiment, the polynucleotide can be DNA. In another embodiment, the polynucleotide can be an antisense polynucleotide. In another embodiment the polynucleotide can be a sense polynucleotide. In another embodiment, the polynucleotide can include at least one nucleotide analog. In another embodiment, the polynucleotide can include a phosphodiester linked 3′-5′ and 5′-3′ polynucleotide backbone. Alternatively, the polynucleotide can include non-phosphodiester conjugations, such as phosphotioate type, phosphoramidate and peptide-nucleotide backbones. In another embodiment, moieties can be linked to the backbone sugars of the polynucleotide. Methods of creating such conjugations are well known to those of skill in the art.

The polynucleotide can be a single-stranded polynucleotide or a double-stranded polynucleotide. The polynucleotide can have any suitable length. Specifically, the polynucleotide can be about 2 to about 5,000 nucleotides in length, inclusive; about 2 to about 1000 nucleotides in length, inclusive; about 2 to about 100 nucleotides in length, inclusive; or about 2 to about 10 nucleotides in length, inclusive.

An antisense polynucleotide is typically a polynucleotide that is complimentary to an mRNA that encodes a target protein. For example, the mRNA can encode a cancer promoting protein i.e., the product of an oncogene. The antisense polynucleotide is complimentary to the single-stranded mRNA and will form a duplex and thereby inhibit expression of the target gene, i.e., will inhibit expression of the oncogene. The antisense polynucleotides of the invention can form a duplex with the mRNA encoding a target protein and will disallow expression of the target protein.

A “functional RNA” refers to a ribozyme or other RNA that is not translated.

A “polynucleic acid decoy” is a polynucleic acid that inhibits the activity of a cellular factor upon binding of the cellular factor to the polynucleic acid decoy. The polynucleic acid decoy contains the binding site for the cellular factor. Examples of cellular factors include, but are not limited to, transcription factors, polymerases and ribosomes. An example of a polynucleic acid decoy for use as a transcription factor decoy will be a double-stranded polynucleic acid containing the binding site for the transcription factor. Alternatively, the polynucleic acid decoy for a transcription factor can be a single-stranded nucleic acid that hybridizes to itself to form a snap-back duplex containing the binding site for the target transcription factor. An example of a transcription factor decoy is the E2F decoy. E2F plays a role in transcription of genes that are involved with cell-cycle regulation and that cause cells to proliferate. Controlling E2F allows regulation of cellular proliferation. For example, after injury (e.g., angioplasty, surgery, stenting) smooth muscle cells proliferate in response to the injury. Proliferation may cause restenosis of the treated area (closure of an artery through cellular proliferation). Therefore, modulation of E2F activity allows control of cell proliferation and can be used to decrease proliferation and avoid closure of an artery. Examples of other such polynucleic acid decoys and target proteins include, but are not limited to, promoter sequences for inhibiting polymerases and ribosome binding sequences for inhibiting ribosomes. It is understood that the invention includes polynucleic acid decoys constructed to inhibit any target cellular factor.

A “gene therapy agent” refers to an agent that causes expression of a gene product in a target cell through introduction of a gene into the target cell followed by expression. An example of such a gene therapy agent would be a genetic construct that causes expression of a protein, such as insulin, when introduced into a cell. Alternatively, a gene therapy agent can decrease expression of a gene in a target cell. An example of such a gene therapy agent would be the introduction of a polynucleic acid segment into a cell that would integrate into a target gene and disrupt expression of the gene. Examples of such agents include viruses and polynucleotides that are able to disrupt a gene through homologous recombination. Methods of introducing and disrupting genes with cells are well known to those of skill in the art.

An oligonucleotide of the invention can have any suitable length. Specifically, the oligonucleotide can be about 2 to about 100 nucleotides in length, inclusive; up to about 20 nucleotides in length, inclusive; or about 15 to about 30 nucleotides in length, inclusive. The oligonucleotide can be single-stranded or double-stranded. In one embodiment, the oligonucleotide can be single-stranded. The oligonucleotide can be DNA or RNA. In one embodiment, the oligonucleotide can be DNA. In one embodiment, the oligonucleotide can be synthesized according to commonly known chemical methods. In another embodiment, the oligonucleotide can be obtained from a commercial supplier. The oligonucleotide can include, but is not limited to, at least one nucleotide analog, such as bromo derivatives, azido derivatives, fluorescent derivatives or a combination thereof. Nucleotide analogs are well known to those of skill in the art. The oligonucleotide can include a chain terminator. The oligonucleotide can also be used, e.g., as a cross-linking reagent or a fluorescent tag. Many common conjugations can be employed to couple an oligonucleotide to another moiety, e.g., phosphate, hydroxyl, etc. Additionally, a moiety may be linked to the oligonucleotide through a nucleotide analog incorporated into the oligonucleotide. In another embodiment, the oligonucleotide can include a phosphodiester linked 3′-5′ and 5′-3′ oligonucleotide backbone. Alternatively, the oligonucleotide can include non-phosphodiester conjugations, such as phosphotioate type, phosphoramidate and peptide-nucleotide backbones. In another embodiment, moieties can be linked to the backbone sugars of the oligonucleotide. Methods of creating such conjugations are well known to those of skill in the art.

Nucleotide and nucleoside analogues are well known in the art. Examples of such nucleoside analogs include, but are not limited to, Cytovene® (Roche Laboratories), Epivir® (Glaxo Wellcome), Gemzar® (Lilly), Hivid® (Roche Laboratories), Rebetron® (Schering), Videx® (Bristol-Myers Squibb), Zerit® (Bristol-Myers Squibb), and Zovirax® (Glaxo Wellcome). See, Physician's Desk Reference, 2005 Edition.

Polypeptides acting as additional bioactive agents attached to the polymers in the invention biodegradable water soluble polymers can have any suitable length. Specifically, the polypeptides can be about 2 to about 5,000 amino acids in length, inclusive; about 2 to about 2,000 amino acids in length, inclusive; about 2 to about 1,000 amino acids in length, inclusive; or about 2 to about 100 amino acids in length, inclusive.

In one embodiment, the bioactive agent polypeptide attached to the polymer in the invention biodegradable water soluble polymer compositions or when used as a tether to another carrier entity can be an antibody. In one embodiment, the antibody can bind to a cell adhesion molecule, such as a cadherin, integrin or selectin. In another embodiment, the antibody can bind to an extracellular matrix molecule, such as collagen, elastin, fibronectin or laminin. In still another embodiment, the antibody can bind to a receptor, such as an adrenergic receptor, B-cell receptor, complement receptor, cholinergic receptor, estrogen receptor, insulin receptor, low-density lipoprotein receptor, growth factor receptor or T-cell receptor. Antibodies attached to polymers (either directly or by a linker) in the invention medical devices can also bind to platelet aggregation factors (e.g., fibrinogen), cell proliferation factors (e.g., growth factors and cytokines), and blood clotting factors (e.g., fibrinogen). In another embodiment, an antibody can be conjugated to an active agent, such as a toxin. In another embodiment, the antibody can be Abciximab (ReoProR)). Abciximab is a Fab fragment of a chimeric antibody that binds to beta(3) integrins. Abciximab is specific for platelet glycoprotein IIb/IIIa receptors, e.g., on blood cells. Human aortic smooth muscle cells express alpha(v)beta(3) integrins on their surface. Treating beta(3) expressing smooth muscle cells may prohibit adhesion of other cells and decrease cellular migration or proliferation, thus reducing restenosis following percutaneous coronary interventions (CPI) e.g., stenosis, angioplasty, stenting. Abciximab also inhibits aggregation of blood platelets.

In one embodiment, the peptide can be a glycopeptide. “Glycopeptide” refers to oligopeptide (e.g. heptapeptide) antibiotics, characterized by a multi-ring peptide core optionally substituted with saccharide groups, such as vancomycin. Examples of glycopeptides included in this definition may be found in “Glycopeptides Classification, Occurrence, and Discovery,” by Raymond C. Rao and Louise W. Crandall, (“Bioactive agents and the Pharmaceutical Sciences” Volume 63, edited by Ramakrishnan Nagarajan, published by Marcal Dekker, Inc.). Additional examples of glycopeptides are disclosed in U.S. Pat. Nos. 4,639,433; 4,643,987; 4,497,802; 4,698,327, 5,591,714; 5,840,684; and 5,843,889; in EP 0 802 199; EP 0 801 075; EP 0 667 353; WO 97/28812; WO 97/38702; WO 98/52589; WO 98/52592; and in J. Amer. Chem. Soc., 1996, 118, 13107-13108; J. Amer. Chem. Soc., 1997, 119, 12041-12047; and J. Amer. Chem. Soc., 1994, 116, 4573-4590. Representative glycopeptides include those identified as A477, A35512, A40926, A41030, A42867, A47934, A80407, A82846, A83850, A84575, AB-65, Actaplanin, Actinoidin, Ardacin, Avoparcin, Azureomycin, Balhimyein, Chloroorientiein, Chloropolysporin, Decaplanin, -demethylvancomycin, Eremomycin, Galacardin, Helvecardin, Izupeptin, Kibdelin, LL-AM374, Mannopeptin, MM45289, MM47756, MM47761, MM49721, MM47766, MM55260, MM55266, MM55270, MM56597, MM56598, OA-7653, Orenticin, Parvodicin, Ristocetin, Ristomycin, Synmonicin, Teicoplanin, UK-68597, UD-69542, UK-72051, Vancomycin, and the like. The term “glycopeptide” or “glycopeptide antibiotic” as used herein is also intended to include the general class of glycopeptides disclosed above on which the sugar moiety is absent, i.e. the aglycone series of glycopeptides. For example, removal of the disaccharide moiety appended to the phenol on vancomycin by mild hydrolysis gives vancomycin aglycone. Also included within the scope of the term “glycopeptide antibiotics” are synthetic derivatives of the general class of glycopeptides disclosed above, included alkylated and acylated derivatives. Additionally, within the scope of this term are glycopeptides that have been further appended with additional saccharide residues, especially aminoglycosides, in a manner similar to vancosamine.

The term “lipidated glycopeptide” refers specifically to those glycopeptide antibiotics that have been synthetically modified to contain a lipid substituent. As used herein, the term “lipid substituent” refers to any substituent contains 5 or more carbon atoms, preferably, 10 to 40 carbon atoms. The lipid substituent may optionally contain from 1 to 6 heteroatoms selected from halo, oxygen, nitrogen, sulfur, and phosphorous. Lipidated glycopeptide antibiotics are well known in the art. See, for example, in U.S. Pat. Nos. 5,840,684, 5,843,889, 5,916,873, 5,919,756, 5,952,310, 5,977,062, 5,977,063, EP 667, 353, WO 98/52589, WO 99/56760, WO 00/04044, WO 00/39156, the disclosures of which are incorporated herein by reference in their entirety.

Anti-inflammatory agents useful for attachment to polymer of the invention compositions include, e.g. analgesics (e.g., NSAIDS and salicyclates), antirheumatic agents, gastrointestinal agents, gout preparations, hormones (glucocorticoids), nasal preparations, ophthalmic preparations, otic preparations (e.g., antibiotic and steroid combinations), respiratory agents, and skin & mucous membrane agents. See, Physician's Desk Reference, 2005 Edition. Specifically, the anti-inflammatory agent can include dexamethasone, which is chemically designated as (11θ,16I)-9-fluoro-11,17,21-trihydroxy-16-methylpregna-1,4-diene-3,20-dione. Alternatively, the anti-inflammatory agent can include sirolimus (rapamycin), which is a triene macrolide antibiotic isolated from Steptomyces hygroscopicus.

Anti-platelet or anti-coagulation agents include, e.g., Coumadin® (DuPont), Fragmin® (Pharmacia & Upjohn), Heparin® (Wyeth-Ayerst), Lovenox®, Normiflo®, Orgaran® (Organon), Aggrastat® (Merck), Agrylin® (Roberts), Ecotrin® (Smithkline Beecham), Flolan® (Glaxo Wellcome), Halfprin® (Kramer), Integrillin® (COR Therapeutics), Integrillin® (Key), Persantine® (Boehringer Ingelheim), Plavix® (Bristol-Myers Squibb), ReoPro® (Centecor), Ticlid® (Roche), Abbokinase® (Abbott), Activase® (Genentech), Eminase® (Roberts), and Strepase® (Astra). See, Physician's Desk Reference, 2005 Edition. Specifically, the anti-platelet or anti-coagulation agent can include trapidil (avantrin), cilostazol, heparin, hirudin, or ilprost.

Trapidil is chemically designated as N,N-dimethyl-5-methyl-[1,2,4]triazolo[1,5-a]pyrimidin-7-amine.

Cilostazol is chemically designated as 6-[4-(1-cyclohexyl-1H-tetrazol-5-yl)-butoxy]-3,4-dihydro-2(1H)-quinolinone.

Heparin is a glycosaminoglycan with anticoagulant activity; a heterogeneous mixture of variably sulfonated polysaccharide chains composed of repeating units of D-glucosamine and either L-iduronic or D-glucuronic acids.

Hirudin is an anticoagulant protein extracted from leeches, e.g., Hirudo medicinalis.

Iloprost is chemically designated as 5-[Hexahydro-5-hydroxy-4-(3-hydroxy-4-methyl-1-octen-6-ynyl)-2(1H)-pentalenylidene]pentanoic acid.

The immune suppressive agent can include, e.g., Azathioprine® (Roxane), BayRho-D® (Bayer Biological), CellCept® (Roche Laboratories), Imuran® (Glaxo Wellcome), MiCRhoGAM® (Ortho-Clinical Diagnostics), Neoran® (Novartis), Orthoclone OKT3® (Ortho Biotech), Prograf® (Fujisawa), PhoGAM® (Ortho-Clinical Diagnostics), Sandimmune® (Novartis), Simulect® (Novartis), and Zenapax® (Roche Laboratories).

Specifically, the immune suppressive agent can include rapamycin or thalidomide. Rapamycin is a triene macrolide isolated from Streptomyces hygroscopicus.

Thalidomide is chemically designated as 2-(2,6-dioxo-3-piperidinyl)-1H-iso-indole-1,3(2H)-dione.

Anti-cancer or anti-cell proliferation agents that can be used as an bioactive agent in the invention compositions include, e.g., nucleotide and nucleoside analogs, such as 2-chloro-deoxyadenosine, adjunct antineoplastic agents, alkylating agents, nitrogen mustards, nitrosoureas, antibiotics, antimetabolites, hormonal agonists/antagonists, androgens, antiandrogens, antiestrogens, estrogen & nitrogen mustard combinations, gonadotropin releasing hormone (GNRH) analogues, progestrins, immunomodulators, miscellaneous antineoplastics, photosensitizing agents, and skin and mucous membrane agents. See, Physician's Desk Reference, 2005 Edition.

Suitable adjunct antineoplastic agents include Anzemet® (Hoeschst Marion Roussel), Aredia® (Novartis), Didronel® (MGI), Diflucan® (Pfizer), Epogen® (Amgen), Ergamisol® (Janssen), Ethyol® (Alza), Kytril® (SmithKline Beecham), Leucovorin® (Immunex), Leucovorin® (Glaxo Wellcome), Leucovorin® (Astra), Leukine® (Immunex), Marinol® (Roxane), Mesnex® (Bristol-Myers Squibb Oncology/Immunology), Neupogen (Amgen), Procrit® (Ortho Biotech), Salagen® (MGI), Sandostatin® (Novartis), Zinecard® (Pharmacia and Upjohn), Zofran® (Glaxo Wellcome) and Zyloprim® (Glaxo Wellcome).

Suitable miscellaneous alkylating agents include Myleran® (Glaxo Wellcome), Paraplatin® (Bristol-Myers Squibb Oncology/Immunology), Platinol® (Bristol-Myers Squibb Oncology/Immunology) and Thioplex® (Immunex).

Suitable nitrogen mustards include Alkeran® (Glaxo Wellcome), Cytoxan® (Bristol-Myers Squibb Oncology/Immunology), Ifex® (Bristol-Myers Squibb Oncology/Immunology), Leukeran® (Glaxo Wellcome) and Mustargen® (Merck).

Suitable nitrosoureas include BiCNU® (Bristol-Myers Squibb Oncology/Immunology), CeeNU® (Bristol-Myers Squibb Oncology/Immunology), Gliadel® (Rhone-Poulenc Rover) and Zanosar® (Pharmacia and Upjohn).

Suitable antibiotics include Adriamycin PFS/RDF® (Pharmacia and Upjohn), Blenoxane® (Bristol-Myers Squibb Oncology/Immunology), Cerubidine® (Bedford), Cosmegen® (Merck), DaunoXome® (NeXstar), Doxil® (Sequus), Doxorubicin Hydrochloride® (Astra), Idamycin® PFS (Pharmacia and Upjohn), Mithracin® (Bayer), Mitamycin® (Bristol-Myers Squibb Oncology/Immunology), Nipen® (SuperGen), Novantrone® (Immunex) and Rubex® (Bristol-Myers Squibb Oncology/Immunology).

Suitable antimetabolites include Cytostar-U® (Pharmacia and Upjohn), Fludara® (Berlex), Sterile FUDR® (Roche Laboratories), Leustatin® (Ortho Biotech), Methotrexate® (Immunex), Parinethol® (Glaxo Wellcome), Thioguanine® (Glaxo Wellcome) and Xeloda® (Roche Laboratories).

Suitable androgens include Nilandron® (Hoechst Marion Roussel) and Teslac® (Bristol-Myers Squibb Oncology/Immunology).

Suitable antiandrogens include Casodex® (Zeneca) and Eulexin® (Schering).

Suitable antiestrogens include Arimidex® (Zeneca), Fareston® (Schering), Femara® (Novartis) and Nolvadex® (Zeneca).

Suitable estrogen and nitrogen mustard combinations include Emcyt® (Pharmacia and Upjohn).

Suitable estrogens include Estrace® (Bristol-Myers Squibb) and Estrab® (Solvay).

Suitable gonadotropin releasing hormone (GNRH) analogues include Leupron Depot® (TAP) and Zoladex® (Zeneca).

Suitable progestins include Depo-Provera® (Pharmacia and Upjohn) and Megace® (Bristol-Myers Squibb Oncology/Immunology).

Suitable immunomodulators include Erganisol® (Janssen) and Proleukin® (Chiron Corporation).

Suitable miscellaneous antineoplastics include Camptosar® (Pharmacia and Upjohn), Celestone® (Schering), DTIC-Dome® (Bayer), Elspar® (Merck), Etopophos® (Bristol-Myers Squibb Oncology/Immunology), Etopoxide® (Astra), Gemzar® (Lilly), Hexylen® (U.S. Bioscience), Hycantin® (SmithKline Beecham), Hydrea® (Bristol-Myers Squibb Oncology/Immunology), Hydroxyurea® (Roxane), Intron A® (Schering), Lysodren® (Bristol-Myers Squibb Oncology/Immunology), Navelbine® (Glaxo Wellcome), Oncaspar® (Rhone-Poulenc Rover), Oncovin® (Lilly), Proleukin® (Chiron Corporation), Rituxan® (IDEC), Rituxan® (Genentech), Roferon-A® (Roche Laboratories), Taxol® (paclitaxol/paclitaxel, Bristol-Myers Squibb Oncology/Immunology), Taxotere® (Rhone-Poulenc Rover), TheraCys® (Pasteur Merieux Connaught), Tice BCG® (Organon), Velban® (Lilly), VePesid® (Bristol-Myers Squibb Oncology/Immunology), Vesanoid® (Roche Laboratories) and Vumon® (Bristol-Myers Squibb Oncology/Immunology).

Suitable photosensitizing agents include Photofrin® (Sanofi).

Specifically, the anti-cancer or anti-cell proliferation agent can include Taxol® (paclitaxol), a nitric oxide-like compound, or NicOX (NCX-4016). Taxol® (paclitaxol) is chemically designated as 5β,20-Epoxy-1,2α4,7β,10β,13α-hexahydroxytax-11-en-9-one 4,10-diacetate 2-benzoate 13-ester with (2R,3S)—N-benzoyl-3-phenylisoserine.

A nitric oxide-like agent includes any bioactive agent that contains a nitric oxide releasing functional group. Suitable nitric oxide-like compounds are S-nitrosothiol derivative (adduct) of bovine or human serum albumin and as disclosed, e.g., in U.S. Pat. No. 5,650,447. See, e.g., David Marks et al., “Inhibition of neointimal proliferation in rabbits after vascular injury by a single treatment with a protein adduct of nitric oxide,” J Clin. Invest. (1995) 96:2630-2638. NCX-4016 is chemically designated as 2-acetoxy-benzoate 2-(nitroxymethyl)-phenyl ester, and is an antithrombotic agent.

It is appreciated that those skilled in the art understand that the bioactive agent or additional bioactive agent useful in the present invention is the bioactive substance present in any of the bioactive agents or agents disclosed above. For example, Taxol® is typically available as an injectable, slightly yellow viscous solution. The bioactive agent, however, is a crystalline powder with the chemical name 5β,20-Epoxy-1,2α,4,7β,10β,13α-hexahydroxytax-11-en-9-one 4,10-diacetate 2-benzoate 13-ester with (2R,3S)—N-benzoyl-3-phenylisoserine. Physician's Desk Reference (PDR), Medical Economics Company (Montvale, N.J.), (53rd Ed.), pp. 1059-1067.

As used herein a “residue of a bioactive agent” is a radical of such bioactive agent as disclosed herein having one or more open valences. Any synthetically feasible atom or atoms of the bioactive agent can be removed to provide the open valence, provided bioactivity is substantially retained when the radical is attached to a residue of a polymer described herein. Based on the conjugation that is desired, those skilled in the art can select suitably functionalized starting materials that can be derived from a bioactive agent using procedures that are known in the art.

The residue of a bioactive agent or additional bioactive agent, as described herein, can be formed employing any suitable reagents and reaction conditions. Suitable reagents and reaction conditions are disclosed, e.g., in Advanced Organic Chemistry, Part B: Reactions and Synthesis, Second Edition, Carey and Sundberg (1983); Advanced Organic Chemistry, Reactions, Mechanisms and Structure, Second Edition, March (1977); and Comprehensive Organic Transformations, Second Edition, Larock (1999).

In certain embodiments, the polymer-bioactive agent conjugation can degrade to provide a suitable and effective amount of free bioactive agent. As will be appreciated by those of skill in the art, depending upon the chemical and therapeutic properties of the bioactive agent, in certain other embodiments, the bioactive agent attached to the polymer performs its therapeutic effect while still attached to the polymer, such as is the case with the “sticky” polypeptides Protein A and Protein G, known herein as “bioligands”, which function while attached to the polymer to hold a target molecule close to the polymer, and the bradykinins and antibodies, which function by contacting (e.g., bumping into) a receptor on a target molecule. Any suitable and effective amount of bioactive agent can be released and will typically depend, e.g., on the specific polymer, bioactive agent, and polymeribioactive agent conjugation chosen. Typically, up to about 100% of the bioactive agent can be released from the polymer by degradation of the polymer backbone as well as the polymer/bioactive agent conjugation. Specifically, up to about 90%, up to 75%, up to 50%, or up to 25% of the bioactive agent can be released from the polymer. Factors that typically affect the amount of the bioactive agent that is released from the polymer is the type of polymer/bioactive agent conjugation, and the nature and amount of additional substances present in the formulation.

The polymer-bioactive agent conjugation can degrade over a period of time to provide time release of a suitable and effective amount of bioactive agent. Any suitable and effective period of time can be chosen. Typically, the suitable and effective amount of bioactive agent can be released in about twenty-four hours, in about seven days, in about thirty days, in about ninety days, or in about one hundred and twenty days. Factors that typically affect the length of time over which the bioactive agent is released from the polymer include, e.g., the nature and amount of polymer, the nature and amount of bioactive agent, the nature of the polymer/bioactive agent conjugation, and the nature and amount of additional substances present in the formulation.

Any suitable size of polymer and bioactive agent can be employed to provide such a water soluble composition. For example, the polymer can have a size of less than about 1×10⁻⁴ meters, less than about 1×10⁻⁵ meters, less than about 1×10⁻⁶ meters, less than about 1×10⁻⁷ meters, less than about 1×10⁻⁸ meters, or less than about 1×10⁻⁹ meters.

The invention composition can degrade to provide a suitable and effective amount of the bioactive agents. Any suitable and effective amount of bioactive agent can be released and will typically depend, e.g., on the specific formulation chosen. Typically, up to about 100% of the bioactive agent can be released from the composition. Specifically, up to about 90%, up to 75%, up to 50%, or up to 25% of the bioactive agent can be released from the composition. Factors that typically affect the amount of the bioactive agent that is released from the composition include, e.g., the nature and amount of polymer, the nature and amount of bioactive agent, and the nature and amount of additional substances present in the composition.

The invention composition can comprise and degrade over a period of time to provide a suitable and effective amount of bioactive agent. Any suitable and effective period of time can be chosen. Typically, the suitable and effective amount of bioactive agent can be released in about one hour, in about six hours, in about twenty-four hours, in about seven days, in about thirty days, in about ninety days, or in about one hundred and twenty days. Factors that typically affect the length of time in which the bioactive agent is released from the composition include, e.g., the nature and amount of polymer, the nature and amount of bioactive agent, and the nature and amount of additional substances present in the composition.

The biological applications of the invention water soluble PEAs, PEURs and PEUs with multiple attachment sites are much broader than those of hydrolytically stable polyethylene glycols (PEGs) with only available two functionalizable end-groups. For example, the invention water soluble PEAs, PEURs and PEUs can be conjugated to various proteins and polynucleotides to form prodrugs for pharmaceutical applications. Modification of small-molecule pharmaceuticals by conjugation to the invention water soluble PEAs and PEURs can be used to improve solubility, enhance control of permeability through biological barriers, increase the half-life in the blood stream, and control release rate of the pharmaceutical from the prodrug.

For use in industrial processing, the invention water soluble PEAs, PEURs and PEUs can be conjugated to enzymes. Such polymer-enzyme conjugates can be used to increase solubility of compounds in water. This feature is useful, for example, to enhance aqueous two-phase partitioning of proteins and in cell purification, to reduce the rate of kidney clearance of industrial by products, and reduce the toxicity of industrial waste products.

In particular, modification of the surface of a compound, a particle, a liposome or a micelle with the invention water soluble polymer composition will cause proteins and cells to reject the modified entity. For example, in one embodiment the invention water soluble polymer composition is applied as a surface modification (e.g., as a coating or by attachment as a tether to a functionalized surface) of a liposome, micelle or polymer particle carrier for a drug or biologic to reduce blood protein adherence to the carrier and so increase blood circulation time of the cargo drug or other biologic.

In another embodiment, molecules of the invention water soluble polymer composition can be attached to the functionalized surface of a polymer particle, liposome or micelle carrier to solubilize the carrier and/or to tether a targeting molecule, such as an antibody, affinity ligand, or cofactor, to such a carrier for biological targeting or signaling. Molecules of the invention water soluble polymer compositions can also be attached to the surface of such a carrier having suitable functional groups to aid in synthesis of biomolecules, affinity ligands and cofactors. For example any carrier having a functionalized surface, such as a polymer particle, the surface of a 96-well tissue culture plate, and the like, can be modified with molecules of the invention water soluble polymer compositions as tethers to aid in controlled synthesis (i.e., residue by residue) of biomolecules, such as polynucleotides and proteins. The synthesis itself can proceed by any method known in the art that occurs in aqueous solution. In addition, protective conjugation of the invention water soluble polymer composition to an individual, soluble biologic can increase half-life of the biologic while maintaining solubility in aqueous conditions.

For example, the invention water soluble polymer composition can be used for surface modification of particles comprising PEA, PEUR or PEU polymers. Methods for attachment of certain water solubilizing molecules to the surface of such particles are described in U.S. patent application Ser. No. 11/344,689, filed Jan. 31, 2007, a copy of which is incorporated herein by reference in its entirety.

The invention water soluble polymer can be used as described herein to increase water solubility of a bioactive agent conjugated thereto by a factor of about 50 fold to about 6,000 fold, or about 100 fold to about 3,000 fold. As shown in illustrative Example 3 below, conjugation of the hydrophobic anti-cancer drug paclitaxel (Taxol) with PEA polymer (Compound 3.3 herein) via pendent hydroxyl groups of the polymer increased solubility of the paclitaxel in water about 5508 times.

The following Examples are meant to illustrate, and not to limit, the invention.

### EXAMPLE 1

This example illustrates synthesis of monomers used in fabrication of invention water soluble polymers.

A. Materials and Methods: Materials: Chemicals Glycerol, 2-O-benzylglycerol, (±)-1-benzylglycerol, glycine, Boc-glycine, trifluoroacetic acid (TFA), p-toluenesulfonic acid monohydrate, benzyl alcohol, adipoyl chloride, glutaryl chloride, succinyl chloride and diglycolyl chloride, 1,6-Hexanediol, p-nitrophenol, triethylamine, 4-N,N-(dimethylamino)pyridine (DMAP), N,N′-dicyclohexylcarbodiimide (DCC), anhydrous N,N-dimethylformamide (DMF), anhydrous dichloromethane (DCM) were purchased from Aldrich Chemicals and used as received. Other anhydrous solvents: ether, ethyl acetate (EtOAc) and tetrahydrofuran (THF) were purchased from Fisher Scientific.

Characterization Procedures: The chemical structures of monomers and polymer were characterized by standard chemical methods. NMR spectra were recorded by a Bruker AMX-500 spectrometer (Numega R. Labs Inc. San Diego, Calif.) operating at 500 MHz for ¹H NMR spectroscopy. Deuterated solvents CDCl₃ or DMSO-d₆ (from Cambridge Isotope laboratories, Inc.) were used with tetramethylsilane (TMS) as and internal standard.

Melting points of synthesized monomers were determined using an automatic Mettler Toledo FP62 Melting Point Apparatus. Thermal properties of synthesized monomers and polymers were characterized using a Mettler Toledo DSC 822e differential scanning calorimeter (DSC). Samples were placed in aluminum pans. Measurements were carried out at a scanning rate of 10° C./min under nitrogen flow.

The number and weight average molecular weights (Mw and Mn) and molecular weight distribution of synthesized polymers were determined by gel permeation chromatography (Model 515, Waters Associates Inc. Milford, USA) equipped with a high pressure liquid chromatographic pump, a Waters 2414 refractory index detector. A 0.1% solution of LiCl in DMAc was used as eluent (1.0 mL/min). Two Styragel HR 5E DMF type columns from Waters were connected and calibrated with polystyrene standards.

B. Methods for Monomer Synthesis.

1). Bis-nucleophiles or bis(α-amino acid)-diol-diesters of general formula (XIII) were synthesized either by DCC technique or by direct condensation of diols with alpha amino acids in the presence of p-toluenesulfonic acid and by azeotropic removal of evolved water. Di-amines were introduced in salt form into a polycondensation reaction, using either TFA or TosOH acids.

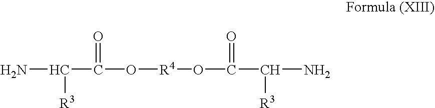

Synthesis of Di-TFA salt of bis(glycine)-1,3-diglyceride (Compound 1.1). Synthesis was conducted using carbodiimide technique and benzyl-protected monomer was introduced into the PEA backbone (FIG. 2). Boc-glycine (5.25 g, 30.0 mmol) was dissolved in dry dichloromethane (50.0 ml) and added 2-O-benzylglycerol (1.82 g, 10.0 mmol) followed by DCC (6.18 g, 30.0 mmol), stirred the mixture for 5 minutes at room temperature. 4-Dimethylaminopyridine (DMAP) (0.24 g, 0.2 mmol) was dissolved in dichloromethane (4.0 mL) and added slowly at 0° C. under argon. The reaction was stirred another 15 minutes at 0° C. and continued at room temperature for 3 days. After complete consumption of compound 2-O-benzylglycerol (TLC, hexane:ethyl acetate in 6:4 volume ratio), the formed urea derivative was removed through glass frit, washed with dichloromethane (3×25 ml), and combined filtrate was concentrated under vacuum. The oily product compound was purified by column chromatography using hexane/ethyl acetate as eluents (at volume ratio of 8:2 then 7:3). All fractions were combined, concentrated and dried, yielding which gave 4.8 g (96.7%) of pure product (Compound 1.1a). Deprotection of Boc-group was conducted in dichloromethane (25 ml) by slowly adding TFA (25 ml) at 0° C., under argon while stirring. After complete addition, the ice bath was removed and stirring was continued for 2 h at room temperature. Consumption of starting material was monitored by TLC (using Hexane:Ethylacetate, in a volume ratio of 6:4). Pouring of the reaction mixture into cold ether yielded a white solid, which was washed with hexanes, filtered, and then washed again with ether (2×20 mL). The compound was dried under vacuum at 35° C. The yield of the purified monomer salt (Compound 1.1) was 85.28% (4.23 g).

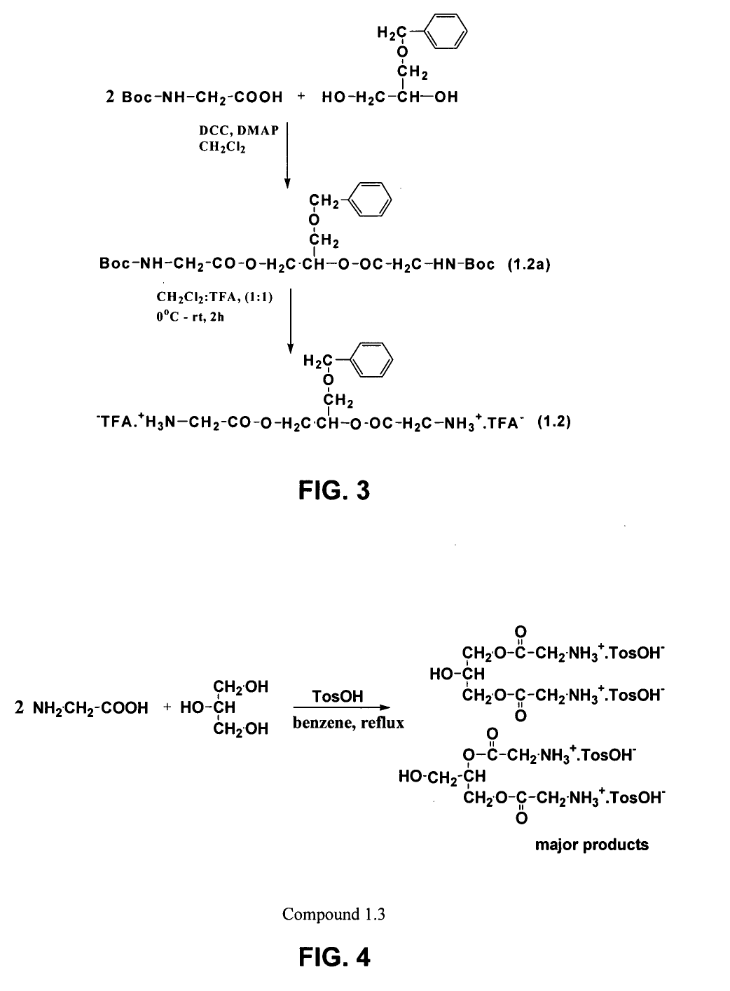

Synthesis of Di-TFA salt of bis-(glycine)-1,2-diglyceride (Compound 1.2) Compound (1.2) was synthesized using a procedure analogous to that described for Compound (1.1), using DCC technique according to the scheme show in FIG. 3. The recrystallization of TFA salt was achieved by dissolving of viscous liquid in a minimum amount of 2-propanol, followed by the addition of diethyl ether in 5× excess. Solvent was decanted and the viscous product was then scratched with a spatula to form a white solid, which was dried in vacuum for about 2 days. The yield of pure monomer salt was 81.3% (15.0 g). ¹H NMR (DMSO-d₆): δ=3.65 (s, 2H), 3.82-3.85 (s, s 4H, —CO—CH₂—NH₃⁺), 4.37 (m, 2H), 4.52 (s, 2H, —CH₂—C₆H₅), 5.29 (s, 1H), 7.32 (m, 5H, Ar) 8.44 (s, broad 6H, —NH₃⁺).

Synthesis of an isomeric mixture of glycerol-bis(glycine)diester ditosylates (Compound 1.3 (FIG. 4): A 1 liter 3-neck round-bottom flask was charged with glycerol (10.0 g, 0.109 mol), p-toluenesulfonic acid monohydrate (46.5 g, 0.244 mol), and glycine (16.7 g, 0.223 mol). Benzene (250 mL) was added while an overhead stirrer was used to ensure good mixing. The reaction continued at reflux for 48 h. A Dean-Stark apparatus was used to collect the water evolved (8.3 mL, 0.462 mol). The reaction mixture was cooled to room temperature and the benzene was decanted from the flask. Ether (100 mL) was used to rinse the resultant hard white solid, which formed on the bottom of reaction flask. The monomer (isomer mixture) as an amorphous, hygroscopic solid was recrystallized from isopropanol/ether twice and dried under vacuum for 48 h at 45° C. Yield; ¹H NMR of resulting monomer mixture shows batch to batch different ratios of 1,2- and 1,3-diesters. Solid phase isomerization of isomer mixture at 50° C. after 7 days, as described by Mank A P J et al. (Chem. Phys. Lipids (1976) 16: 107-114), was not observed. Separation of di-acid salt isomers or their free diamines by column chromatography or vacuum distillation failed.

Synthesis of Di-p-toluenesulfonic acid salt of bis-glycine-1,4-anhydroerythritol diester (Compound 1.3) Using a previously published method (Gomurashvili, Z, et al. J.M.S.—Pure Appl. Chem. (2000), 37:215-227) Compound 1.3 was synthesized as shown in the following scheme: 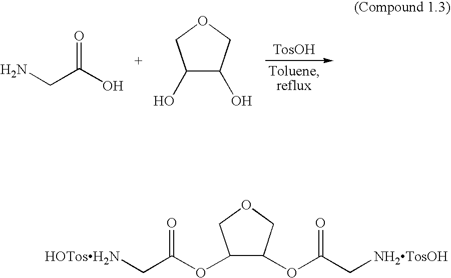
Glycine (24.92 g, 0.332 mol), p-toluenesulfonic acid monohydrate (69.46 g, 0.365 mol) and 1,4-anhydroerythritol (17.28 g, 0.165 mol) in 300 mL of toluene were charged in a flask equipped with a Dean-Stark apparatus and overhead stirrer. The heterogeneous reaction mixture was heated to reflux for about. 24 h until 12.6 mL (0.697 mol) of water evolved. The reaction mixture was then cooled to room temperature, filtered, washed with acetone and recrystallized twice from a mixture methanol/2-propanol at 1:2 volume ratio. White powder with mp=224° C. in 62% yield was collected. ¹H NMR (DMSO-d₆): δ=2.29 (s, 6H, Ar—CH₃), 3.76-3.78 (d, d, 2H, O—CH₂—CH), 3.90 (s, 4H, —CO—CH₂—NH₃⁺), 3.97-4.00 (d, d 2H, O—CH₂—CH), 5.45 (s, 2H, O—CH═), 7.13 (d, 4H, Ar), 7.51 (d, 4H, Ar), 8.28 (s, 6H, —NH₃⁺).

2). Bis-electrophiles: Di-p-nitrophenyl esters (general Formula XIV) of di-acids (adipic, glutaric, succinic, diglycolic acids) were prepared by reaction of di-acid chlorides with p-nitrophenol.

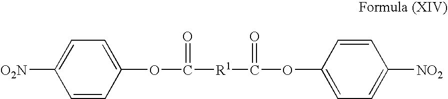

Synthesis of di-p-nitrophenyl ester of diglycolic acid The following exemplary procedure illustrates synthesis of di-p-nitrophenyl ester of diglycolic acid. Triethylamine (61.6 mL, 0.442 mol) was added to a stirring solution of p-nitrophenol (61.5 g, 0.442 mol) in dry acetone (350 mL). The solution was cooled down to 4° C. and diglycolyl chloride (25 mL, 0.21 mol) was added drop-wise to the solution over 30 min under argon. Then the cooling bath was removed and stirring was continued overnight at room temperature. The reaction mixture was diluted with water (450 mL) and stirred for 10 min. The resultant solid was collected by filtration and washed, first with 0.1 N HCl water solution (500 mL) and then with water (500 mL). The resulting di-ester was recrystallized in acetone and then dried at 60° C. under vacuum for 20 h to obtain 32.8 g of product. The filtrate was kept at 4° C. for three days to obtain an additional 11.4 g of product. The combined total yield was 44.2 g (55.9%) with mp 166.8° C., lit. 166-167° C. (Zimmer, H et al., J. Org. Chem. (1975) 40:2901-06). ¹H NMR (DMSO-d₆): δ=4.68 (s, 4H), 7.52 (d, J=7.2 Hz, 4H), 8.34 (d, J=7.2 Hz, 4H).

Other di-esters of di-acids that were synthesized for fabrication of the invention water soluble polymers were:

1. Di-p-nitrophenyl adipate, (Formula IV, wherein R¹=(CH₂)₄): Recrystallized from acetone, yield 85%. mp=123° C. ¹H NMR (DMSO-d₆): δ=1.77 (q, 4H), 2.73 (q, 4H), 7.45 (d, 4H), 8.30 (d, 4H);

2. Di-p-nitrophenyl glutarate, (Formula IV, wherein R¹=(CH₂)₃): Recrystallized from acetone, yield 82%, mp=140° C. ¹H NMR (DMSO-d₆): δ=2.03 (q, 2H), 2.81 (t, 4H), 7.47 (d, 4H), 8.31 (d, 4H); and

3. Di-p-nitropehnyl succinate, (Formula IV, wherein R¹=(CH₂)₂: Synthesis was carried out at −40° C. for 4 h and then slowly warmed up to room temperature. Recrystallized from acetonitrile, mp=183.0° C., mp. lit=180-182° C. (Katsarava R. D. et al. Izv. Akad. N. Gruz. SSR. Khim Ser. (1982), 8(2):102-109); ¹H NMR (DMSO-d₆): δ=3.06 (m, 4H), 7.43 (d, 4H), 8.31 (d, 4H).
Bis-electrophiles (compound XV) useful for synthesis of PEUR polymer of formula (IV and V) were prepared in a similar manner as is described in U.S. Pat. No. 6,503,538 B1 supra; Bis-chloroformate of formula (XV) is prepared by reaction of diol (1,3-propanediol, 1,4-anhydroerythritol) with 2 equiv. of p-nitrophenyl chloroformate in the presence of tertiary amine as acid acceptor. 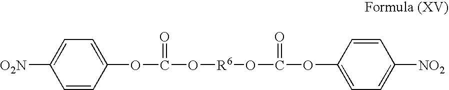
As bis-nucleophile for PEU polymer synthesis (formulas VI and VII), monomeric, dimeric or trimeric phosgene can be used. Polycondensation reaction with monomer XIII generally will be carried out in interfacial manner, for example in water and dichloromethane system, as is known by those skilled in the art.

### EXAMPLE 2

### Synthesis of Polymers by Solution Polycondensation

PEAs were synthesized according to a method previously described by Arabuli, N, et al. (Macromol. Chem. Phys. (1994), 195:2279-2289). Briefly, salts of di-amines were reacted with active di-esters of aliphatic di-acids in the presence of organic base as shown in scheme below: 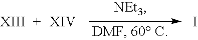
The reactions were mainly carried out in DMF at 60° C., for 24 h. The PEAs formed were purified thoroughly by multiple re-precipitations. Polymers with pending benzylated hydroxyls were then subjected to Pd mediated hydrogenolysis.

The general synthetic procedure is as described as follows for synthesis of benzylated-precursor of polymer Compound 3.2.1: 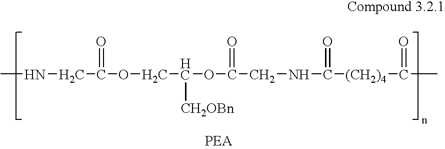
Compound 1.1 (12.902 g, 24.6 mmol) (di-TFA salt of bis(glycine)-1,3-diglyceride) and bis p-nitrophenyl adipate (9.555 g, 24.6 mmol) were weighed into a 100 mL single neck round bottom flask. Triethylamine (7.54 ml) and 13.0 ml of dry DMF were added to the reaction flask and heated to 60° C., for 24 hours. The reaction was then cooled to room temperature, diluted with 20 mL of DMF and, under vigorous stirring, precipitated in 500 mL of water. The polymer formed was re-dissolved in DMF and precipitated 3× in cold methanol. A yield of product white polymer 7.5 g (75.3% yield) was collected; Mw=77 kDa, Mn=51 kDa, Mw/Mn=1.50, Tg=31° C. ¹H NMR (DMSO-d₆): δ=1.48 (q, 4H), 2.12 (q, 4H), 3.81 (m, 5H), 4.11-4.15 (m, 2H), 4.19-4.22 (m, 2H), 4.59 (s, 2H), 7.27 (m, 1H), 7.34 (s, 4H), 8.25 (t, 2H).

Synthesis of PEA (Compound 3.1.1) 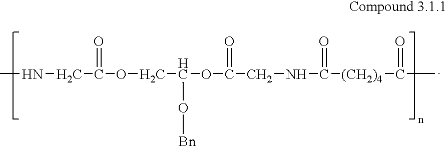
Mw=16 kDa, Mw/Mn=1.52; ¹H NMR (DMSO-d₆): δ=1.49 (s, 4H), 2.13 (s, 4H), 3.59 (d, d, 2H), 3.76-3.85 (m, 4H), 4.22 (m, 2H), 4.90 (d, d, 2H), 5.16 (m, 1H), 7.32 (m, 5H), 8.25 (t, 2H).
Deprotection of Benzyl-Protected PEAs:

De-protection of the benzylated groups by catalytic hydrogenation yielded hydroxyl-bearing PEAs. The catalyst used for hydrogenation was Pd-black and the operation was conducted either under hydrogen atmosphere or in the presence of formic acid. Deprotection of benzylated-groups from primary hydroxyls proceeded smoothly at room temperature.

A typical procedure was as follows: 1.78 g of PEA Compound 3.2.1 was dissolved in 15 mL of DMF and diluted with 15 mL of methanol to which was added 880 mg of Pd Black and 1.78 mL formic acid. The reaction mixture was stirred for 18 h, centrifuged to remove solids, filtered, and then the polymer-containing solution was poured into 300 mL of ether to precipitate the polymer. The ¹H NMR spectrum of the de-protected PEAs showed disappearance of benzyl proton signals (7.3 and 5.2 ppm). GPC spectra recorded the presence of the predicted macromolecule and proved that no chain cleavage occurred.

Hydrogenolysis of PEAs with benzyl-protected secondary hydroxyls was more challenging: no deprotection occurred at room temperature even when a higher ratio of Pd to polymer (1:1 by weight) was applied, neither in the presence of formic acid or free hydrogen. When the conditions described in the literature were used: the solvent system DMF/MeOH (1:1 by volume) at 60° C., ([Wang X L, et al. J. Polym. Sci. Part A: Polym. Chem., (2002) 40:70-75) the PEA polymer was cleaved and after 24 h only oligomers were detected when GPC was used.

Better results were achieved in DMF/ethylacetate, (1:1 volume ratio), at 60° C. to 70° C.: 75% of Benzyl-protecting groups were cleaved off after 8 h at 60° C. and after 24 h no aromatic groups were observed by ¹H NMR. The polymer solution was centrifuged to remove solid catalyst, filtered through glass filter and poured into 300 mL of ether to precipitate the polymer in 78% yield. The resulting PEA Compound 3.1 had a melting endotherm on DSC trace: Tm=123° C. and Tg=8° C.

Polycondensation of PEA Compound 3.5 Using the above procedure, the polymer product yielded was 68%. Mw=23 kDa, Mw/Mn=1.27, Tg=30-C. ¹H NMR (DMSO-d₆): δ=2.40 (t, 4H), 3.67-3.70 (d, d, 2H), 3.84-3.87 (m, 4H), 3.94-3.97 (d, d, 2H), 5.30 (q, 2H), 8.33 (t, 2H).

Polycondensation of PEA Compound 3.3 containing mixed primary and secondary hydroxyls. Since it is extremely hygroscopic, the monomer was weighed into the prepared reaction flask. The flask, along with the adipate monomer, was dried in a vacuum oven for 24 h at 40° C. Di-p-nitrophenol adipate (15.7363 g, 40.523 mmol) was added to the reaction flask containing the glycerol-di-glycine-diester di-tosylate monomer mixture (22.312 g, 40.523 mmol). DMF (21.3 mL, 1.2M) and triethylamine (12.4 ml, 89.151 mmol) were then added to the reaction flask. The reaction was stirred at 60° C. for 24 h. The resulting polymer solution was diluted with 20 mL of DMF and precipitated in acetone. The solid was collected and dissolved again in DMF and repeatedly re-precipitated in cold acetone. Yields of polymer varied from 35 to 70% depending on molecular weight. PEAs of the type of Compound 3.3 have achieved molecular weight range of 18,000-82,000 with polydispersity 1.8-2.5 as determined by GPC.

### EXAMPLE 3

### Synthesis of PEA-Taxol Conjugate

Paclitaxel (Taxol) was attached to PEA through a degradable (ester) conjugation, to ensure that active drug will be released from the polymeric carrier. First, Taxol was linked at the 2′-position with succinic acid, which further played a role of linker between the PEA and the drug.

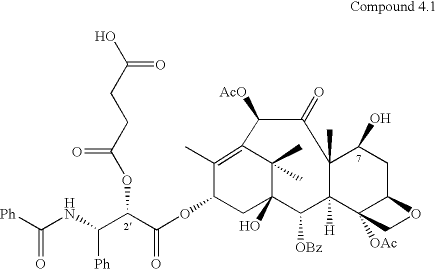

Synthesis of Taxol-2′-hemisuccinate (Taxol-SA) (Compound 4.1) The synthetic procedure followed was as previously published (Yi Luo and Glenn D. Prestwich, Bioconjugate Chem. (1999) 10:755-763). Taxol (90 mg, 105 μmol), succinic anhydride (12.6 mg, 126 μmol, 1.2 equiv) and pyridine (148 μmol, 1.4 equiv) in dichloromethane (4.3 mL) was stirred at room temperature for 3 days. The solvent was removed on a rota-evaporator and the residue was stirred with water (10 mL) for 20 min and filtered over a sintered funnel. The solid was dissolved in acetone (7 mL) and precipitated by addition of water (5 mL). The precipitate was filtered and washed with water (5 mL), then dried under vacuum at 40° C. overnight to obtain 85 mg of solid (85% yield). ¹H NMR and MS were identical to those reported in literature.

Conjugation of Taxol with PEA (PEA-Taxol): PEA polymer (Compound 3.3) (200 mg, M_{w}=16 kDa, repeating unit FW=317) was dried under vacuum at 40° C. for 48 h, then dissolved in DMF (0.5 mL) and dried over molecular sieves for 24 h before transfer into a glass vial containing Taxol-2′-hemisuccinate (20 mg, 21 μmol). The molecular sieves were rinsed with an additional 0.5 ml of DMF and the wash product was added to a reaction vial. To this vial were added DCC (4.28 mg, 21 μmol), 1-hydroxybenzotriazole (2.8 mg, 21 μmol, MW=135.12), DMAP (2.5 mg, 21 μmol) and 1 mL pyridine and the mixture was stirred at room temperature for five days. The reaction mixture was precipitated from acetone (20 mL).

The residue was twice re-dissolved in DMF (1 mL) and precipitated from acetone (20 mL). The precipitate was then dried under vacuum overnight to yield 64 mg of solid (29% yield). According to the results of ¹H NMR assay, the loading of Taxol on PEA was 5.91% w/w and GPC measurement showed increased molecular weight of the conjugate to 34 kDa. Preliminary UV measurements showed that PEA-Taxol prodrug has about 5508 times more solubility in water (1377 μg/mL) than Taxol. (According to M. Vyas et al. Bioorg. Med. Chem. Lett. (1993) 3:1357-1360, free Taxol has solubility in water of 0.25 μg/mL.) The suggested chemical structure of the PEA-Taxol conjugate on secondary hydroxyl is shown below:

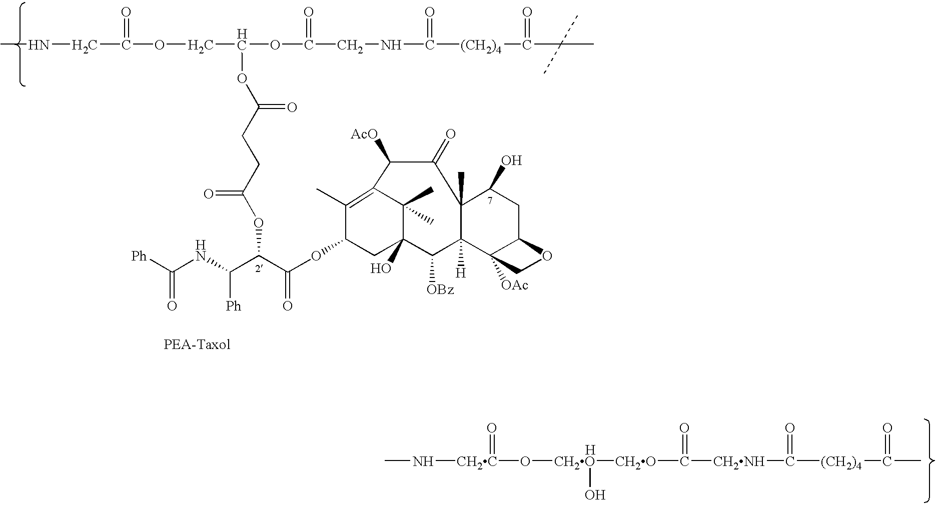

### EXAMPLE 4

### In Vitro Cell Culture Cytotoxicity

This Example illustrates the ability of the water soluble PEA-Taxol conjugate prepared in Example 3 to deliver to test cells cytotoxic amounts of Taxol. In vitro assays were conducted using endothelial cells and smooth muscle cells.

To determine the cytoxicity of the PEA-Taxol conjugate, 1000 cells/well were plated into 96-well tissue culture plates and placed in CO₂ incubator at 37° C., for 24 hours to allow for cell attachment. The cells were then fed again with fresh medium (Endothelial Growth Media, EGM-2 BulletKit, Cambrex) and increasing doses of the test substrates were introduced at the following concentrations: 0.1, 0.5, 1, 2, 8 and 40 ng/ml in triplicate. The substances tested in these experiments were PEA alone, PEA-Taxol conjugate, Taxol-SA, Taxol alone, a DMSO vehicle matching the highest concentration of DMSO present in the assay, and media alone as a positive control. The final concentrations of the PEA-Taxol and PEA are based on the final Taxol concentrations tested in each well (˜6% loading of Taxol). The percent cell viability was then determined at 24, 48 and 72 hours following exposure to the test substances, using a standard ATP assay (ViaLight Plus assay kit, Cambrex).

TABLE 1
% Cell Viability after 72 hour exposure

| | Taxol [ng/ml] | | | | | |
|---|---|---|---|---|---|---|
| | 40 | 8 | 2 | 1 | 0.5 | 0.1 |
| Taxol | 15.5% | 38.6% | 90.0% | 113.6% | 122.6% | 125.8% |
| Taxol-SA | 17.4% | 65.8% | 115.7% | 136.1% | 137.2% | 133.6% |
| PEA-Taxol | 48.4% | 85.4% | 129.0% | 135.2% | 132.8% | 99.3% |
| PEA | 131.9% | 133.5% | 137.9% | 133.9% | 128.5% | 99.3% |
| DMSO-con | 126.5% | | | | | |
| Media-con | 100% | | | | | |

Table 1 shows the data from a representative assay examining the percent cell viaility of endothelial cells exposed to the PEA-Taxol conjugate. Similar results were found when the same test articles were incubated with the smooth muscle cells.

Conjugation of the succinic acid to the Taxol does not appear to change the cytotoxicity of the Taxol-SA to the cells. The PEA Compound 3.3 alone did not show any toxicity to the cells at any of the concentrations used in these assays. Finally, these data demonstrate that the PEA-Taxol conjugate is being delivered to the endothelial cells based on decreasing cell viability with increasing Taxol concentration.

Although the invention has been described with reference to the above examples, it will be understood that modifications and variations are encompassed within the spirit and scope of the invention. Accordingly, the invention is limited only by the following claims.

What is claimed is:

1. A composition comprising at least one of the following:
a PEA polymer having a chemical formula described by general structural formula (I), 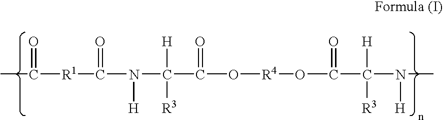
wherein n ranges from about 5 to about 150; R¹ is independently selected from (C₂-C₄) alkylene or CH₂OCH₂; R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻; (CH₂)₂COO⁻; and R⁴ is independently selected from the group consisting of CH₂CH(OH)CH₂ or CH₂CH(CH₂OH), bicyclic-fragments of 1,4:3,6-dianhydrohexitols of structural formula (II), residues of 1,4-anhydroerythritol and combinations thereof, 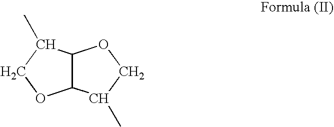
or a PEA polymer having a chemical formula described by structural formula (III): 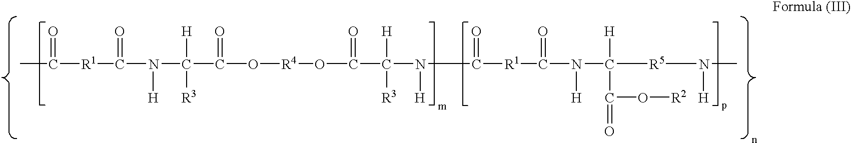
wherein n ranges from about 5 to about 150, m ranges about 0.1 to 0.9; p ranges from about 0.9 to 0.1; R¹ is independently selected from (C₂-C₄) alkylene or CH₂OCH₂; each R² is independently hydrogen, or a protecting group; the R³s in individual units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH); bicyclic-fragments of 1,4:3,6-dianhydro-hexitols of structural formula (II), residues of 1,4-anhydroerythritol and combinations thereof; and R⁵ is independently selected from the group consisting of (C₁-C₄) alkyl;
or a poly(ester urethane) (PEUR) polymer having a chemical formula described by structural formula (IV), 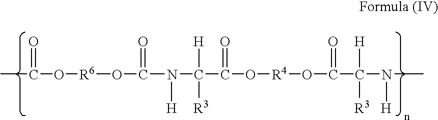
wherein n ranges from about 5 to about 150; wherein R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂, or CH₂CH(CH₂OH), bicyclic-fragments of 1,4:3,6-dianhydrohexitols of structural formula (II), residues of 1,4-anhydroerythritol, and combinations thereof; and R⁶ is independently selected from the group consisting of CH₂CH(OH)CH₂, CH₂CH(CH₂OH), residues of 1,4-anhydroerythritol, and combinations thereof;
or a PEUR polymer having a chemical structure described by general structural formula (V), 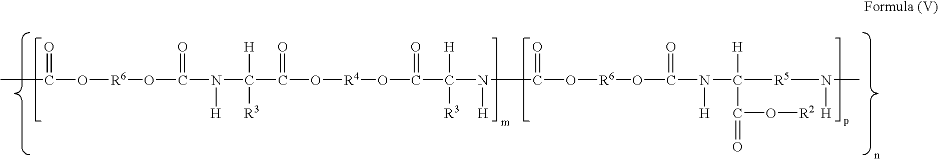
wherein n ranges from about 5 to about 150, m ranges about 0.1 to about 0.9: p ranges from about 0.9 to about 0.1; R² is independently selected from hydrogen or a protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; R⁴ is independently selected from the group consisting of CH₂CH(OH)CH₂ or CH₂CH(CH₂OH), bicyclic-fragments of 1,4:3,6-dianhydrohexitols of structural formula (II), residues of 1,4-anhydroerythritol, and combinations thereof; R⁶ is independently selected from the group consisting of CH₂CH(OH)CH₂, or CH₂CH(CH₂OH), residues of 1,4-anhydroerythritol, and combinations thereof; and R⁵ is independently selected from the group consisting of (C₁-C₄) alkyl;
or a poly(ester urea), (PEU) polymer having a chemical formula described by general structural formula (VI), 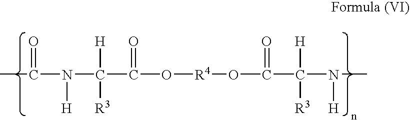
wherein n is about 10 to about 150; each R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, and combinations thereof; and R⁴ is independently selected from the group consisting of CH₂CH(OH)CH₂ or CH₂CH(CH₂OH), residues of 1,4-anhydroerythritol, and combinations thereof;
or a PEU polymer having a chemical formula described by structural formula (VII), 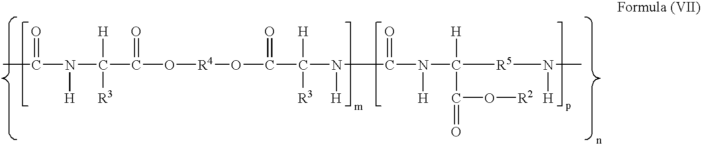
wherein m is about 0.1 to about 1.0; p is about 0.9 to about 0.1; n is about 10 to about 150; each R² is independently hydrogen, or protecting group; the R³s in individual n units are independently selected from the group consisting of hydrogen, CH₂OH, CH(OH)CH₃, (CH₂)₄NH₃⁺, (CH₂)₃NHC(═NH₂⁺)NH₂, 4-methylene imidazolinium, CH₂COO⁻, (CH₂)₂COO⁻, or combinations thereof; R⁴ is independently selected from the group consisting of CH₂CH(OH)CH₂ or CH₂CH(CH₂OH), residues of 1,4-anhydroerythritol, and combinations thereof; and R⁵ is independently selected from the group consisting of (C₁-C₄) alkyl, and
wherein the composition is biodegradable and water soluble.

2. The composition of claim 1, further comprising counter-ions associated with the polymer.

3. The composition of claim 1, further comprising a protecting group bound to the polymer.

4. The composition of claim 1, wherein the polymer comprises at least one pendent hydrophilic group per repeat unit of the polymer.

5. The composition of claim 1, wherein the polymer comprises at least one charged α-amino acid.

6. The composition of claim 1, wherein the polymer comprises at least one pendant polar, but uncharged, primary or secondary hydroxyl group per repeat unit.

7. The composition of claim 1, wherein the R³s comprise (CH₂)₄NH₃⁺).

8. The composition of claim 1, wherein the R³s comprise (CH₂)₃NHC(═NH₂⁺)NH₂).

9. The composition of claim 1, wherein the R³s comprise 4-methylene imidazolinium ion.

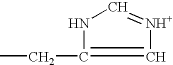

10. The composition of claim 1, wherein the R³s comprise CH₂COO⁻.

11. The composition of claim 1, wherein the R³s comprise (CH₂)₂COO⁻.

12. The composition of claim 1, wherein the polymer comprises at least one pendant polar and positively or negatively charged group per repeat unit.

13. The composition of claim 2, wherein at least one of the counter-ions is a bioactive agent.

14. The composition of claim 1, wherein the polymer comprises at least one pendant polar, but uncharged, primary or secondary hydroxyl group and at least one pendant positively or negatively charged group per repeat unit.

15. The composition of claim 1, wherein the polymer comprises at least two different amino acids.

16. The composition of claim 1, further comprising at least one bioactive agent conjugated to the polymer for controlled release over time.

17. The composition of claim 16, wherein the bioactive agent is conjugated to at least one amino group or activated ester group of the polymer.

18. The composition of claim 1, further comprising a bioactive agent conjugated to the polymer and solubility of the composition in aqueous solution is from 50 fold to 5000 fold greater than that of the bioactive agent alone in aqueous solution.

19. The composition of claim 1, wherein the composition further comprises a bioactive agent and a particle, liposome, or micelle with the bioactive agent tethered to the particle, liposome or micelle via the polymer to enhance water solubility of the bioactive agent.

20. The composition of claim 1, further comprising a bioactive agent conjugated to at least one pendent hydrophilic functional group of the polymer to form a prodrug for controlled release of the bioactive agent.

21. The composition of claim 20, wherein the bioactive agent is conjugated to at least one pendent hydrophilic functional group of the polymer per repeat unit thereof.

22. The composition of claim 20, wherein a dispersion of the composition in aqueous solution spontaneously forms a free-swimming, fully soluble nanoparticle with the bioactive agent sequestered therein.

23. The composition of claim 20, wherein solubility of the composition in deionized water is from 50 to 5000 times greater than that of the bioactive agent alone therein.

24. A method for delivering a bioactive agent to a subject in a controlled manner, said method comprising administering to the subject a composition of claim 1 to which is conjugated at least one bioactive agent to deliver the bioactive agent to the subject in a controlled manner over time.

25. The method of claim 24, wherein the at least one bioactive agent is conjugated with a pendant reactive group of the polymer or via an amine or carboxylic end-group of the polymer macrochain.

26. The method of claim 24, wherein circulation half-life of the bioactive agent is increased.

27. The method of claim 24, wherein water solubility of the bioactive agent is thereby increased.

28. The method of claim 24, further comprising, prior to the administering, attaching the composition to the surface of a particle, a liposome or a micelle.

29. A method for increasing water solubility of a bioactive agent comprising conjugating at least one bioactive agent to a pendant reactive group or via an amine or carboxylic end-group of the polymer macrochain of the composition of claim 1 to increase water solubility of the bioactive agent in a prodrug so formed as compared with that of the bioactive agent alone.

30. The method of claim 29, wherein the prodrug comprises at least one α-amino acid that is charged in deionized water.

31. The method of claim 29, wherein water solubility of the bioactive agent is increased from about 50 fold to about 5000 fold.

32. The method of claim 29, wherein the polymer in the prodrug comprises at least one pendant polar and positively or negatively charged group per repeat unit in deionized water.

33. The method of claim 29, wherein the polymer in the prodrug comprises at least one pendant polar but uncharged primary or secondary hydroxyl group and at least one pendant positively or negatively charged group per repeat unit in deionized water.
